(19) United States
(12) Patent Application Publication
(10) Pub. No.: US 2022/0280407 A1
(43) Pub. Date: Sep. 8, 2022

(54) METHOD FOR TREATING KERATIN MATERIAL, COMPRISING THE USE OF AN ORGANIC C1-C6-ALKOXY-SILANE AND AN AMINO ACID AND/OR AN AMINO ACID DERIVATIVE

(71) Applicant: Henkel AG & Co. KGaA, Duesseldorf (DE)

(72) Inventors: Marc NOWOTTNY, Moenchengladbach (DE); Juergen SCHOEPGENS, Schwalmtal (DE); Carsten MATHIASZYK, Essen (DE); Phillip JAISER, Langenfeld (DE); Ulrike SCHUMACHER, Duesseldorf (DE); Caroline KRIENER, Duesseldorf (DE); Claudia KOLONKO, Remscheid (DE); Torsten LECHNER, Langenfeld (DE); Gabriele WESER, Neuss (DE); Jing HODES, Hagen (DE)

(73) Assignee: Henkel AG & Co. KGaA, Duesseldorf (DE)

(21) Appl. No.: 17/631,775

(22) PCT Filed: Jun. 8, 2020
(86) PCT No.: PCT/EP2020/065776

§ 371 Date: Jan. 31, 2022

(30) Foreign Application Priority Data
Aug. 1, 2019 (DE) 10 2019 211 510.1

(51) Int. Cl. A61K 8/58; A61Q 5/10; A61K 8/44; A61K 8/64; A61K 8/65

(52) U.S. Cl. CPC A61K 8/585; A61Q 5/10; A61K 8/44; A61K 8/645; A61K 8/65; A61K 2800/95; A61K 2800/4324; A61K 2800/884

(57) ABSTRACT

It is an object of the present disclosure is to provide a method for treating keratinous material, in particular human hair, wherein on the keratinous material are applied:
a first composition (A) comprising:
   (A1) one or more organic C₁-C₆ alkoxy silanes and/or condensation products thereof, and
a second composition (B) comprising
   (B1) at least one compound selected from the group of amino acids and protein hydrolysates,
      where
the total content of all polymers contained in the composition (A)—based on the total weight of the composition (A)—is below about 0.3% by weight, and
the total content of all polymers contained in the composition (B)—based on the total weight of the composition (B)—is below about 0.3% by weight.

### CROSS-REFERENCE TO RELATED APPLICATIONS

This application is a U.S. National-Stage entry under 35 U.S.C. § 371 based on International Application No. PCT/EP2020/065776, filed Jun. 8, 2020, which was published under PCT Article 21(2) and which claims priority to German Application No. 10 2019 211 510.1, filed Aug. 1, 2019, which are all hereby incorporated in their entirety by reference.

### TECHNICAL FIELD

The present application is in the field of cosmetics and relates to a method for the treatment of keratinic material, in particular human hair, which comprises the use of two compositions (A) and (B). The composition (A) is a preparation comprising at least one C₁-C₆ organic alkoxysilane, and the composition (B) includes at least one compound (B1) selected from the group of amino acids and protein hydrolysates. Here, the two compositions (A) and (B) are exemplified in that their polymer content is each limited to a certain maximum content.

A second object of the present disclosure is a multi-component packaging unit (kit-of-parts) for dyeing keratinous material, which comprises, separately packaged in two packaging units, the two compositions (A) and (B) described above.

### BACKGROUND

The change in shape and color of keratin fibers, especially hair, is a key area of modern cosmetics. To change the hair color, the expert knows various coloring systems depending on coloring requirements. Oxidation dyes are usually used for permanent, intensive dyeings with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeings obtained with direct dyes have a shorter shelf life and quicker wash ability. Dyeing with direct dyes usually remain on the hair for a period of between 5 and about 20 washes.

The use of color pigments is known for short-term color changes on the hair and/or skin. Color pigments are understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

EP 2168633 B1 deals with the task of producing long-lasting hair colorations using pigments. The paper teaches that when a combination of pigment, organic silicon compound, hydrophobic polymer and a solvent is used on hair, it is possible to produce colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these alkoxy silanes or their hydrolysis or condensation products are applied to keratinous material, a film or coating forms on the keratinous material, which completely coats the keratinous material and, in this way, strongly influences the properties of the keratinous material. Areas of application include permanent styling or permanent shape modification of keratin fibers. In this process, the keratin fibers are mechanically shaped into the desired form and then fixed in this form by forming the coating described above. Another particularly suitable application is the coloring of keratin material; in this application, the coating or film is produced in the presence of a coloring compound, for example a pigment. The film colored by the pigment remains on the keratin material or keratin fibers and results in surprisingly wash-resistant colorations.

The great advantage of the alkoxy silane-based dyeing principle is that the high reactivity of this class of compounds enables very fast coating. This means that good coloring results can be achieved even after short application periods of just a few minutes.

In prior state of the art documents, such as EP 2168633 B1, film-forming polymers are often used in the hair dyeing process to increase fastness properties. Although the use of these polymers brings certain advantages in terms of increasing wash fastnesses, there are also various disadvantages associated with their use. The polymers used in the dyeing process of EP 2168633 B1 are deposited on the keratin material due to their film-forming properties, so that a second class of substances is applied in addition to the organic silicon compounds, resulting in a coating or overlay on the keratin material. This increased film formation, or the formation of very thick films can weigh down the hair in a detrimental way, so that in the worst case the user perceives a collapsed hairstyle, little volume and an unpleasant feel on his hair. In addition, it has been found that the presence of the polymer can cause problems when the staining is repeated. Whereas very satisfactory color intensities could still be achieved with the first application of this dyeing system, it turned out with the subsequent application that remaining polymer radicals negatively influenced the color intensity of the second dyeing. For this reason, there is still a great need for pigment dyeing systems that lead to intensive and wash-resistant dyeings even without the use of polymers and that are not associated with a reduction in color intensity even with repeated use.

It was therefore the task of the present application to find a process for the treatment of keratinous material which can also be used in the dyeing of hair and in this connection shows improvements regarding color intensity and fastness to washing. With repeated application, the performance of dyeing should still be exactly as high as with the first application. The color intensities and wash fastnesses should be improved compared to the colorations that can be achieved so far with the formulations known from the prior art. Due to the previously described disadvantages, the presence of polymers should be avoided to solve this task.

### BRIEF SUMMARY

Methods of treating keratinous material and kits-of-parts for the same are provided. In an exemplary embodiment, a method includes applying a first composition (A) and a second composition (B) to the keratinous material. The first composition (A) comprises one or more organic C₁-C₆ alkoxy silanes (A1) and/or condensation products thereof. The second composition (B) comprises a compound (B1) selected from the group of amino acids, protein hydrolysates, or combinations thereof. The total content of polymers in the first composition (A) is below about 0.3 weight percent, and the total content of polymers in the second composition (B) is below about 0.3 weight percent.

A Kit-of-parts is provided in another embodiment. The kit of parts includes a first container containing a first composition (A) and a second container containing a second composition (B). The first composition (A) comprises one or more organic C₁-C₆ alkoxy silanes (A1) and/or condensation products thereof. The second composition (B) comprises a compound (B1) selected from the group of amino acids, protein hydrolysates, or combinations thereof. The total content of polymers in the first composition (A) is below about 0.3 weight percent, and the total content of polymers in the second composition (B) is below about 0.3 weight percent

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has been found that this task can be fully solved if the keratin material is treated in a process in which two compositions (A) and (B) are applied to the keratin material. Here, the first composition (A) contains at least one organic C₁-C₆ alkoxy silane and/or its condensation product, and the second composition (B) is exemplified by its content of at least one compound selected from the group of amino acids and protein hydrolysates. To achieve correspondingly good dyeing performances even with repeated use, the presence of a polymer was not required in either composition (A) or composition (B).

A first object of the present disclosure is a method for treating keratinous material, in particular human hair, wherein on the keratinous material are applied:

a first composition (A) comprising:
(A1) one or more organic C₁-C₆ alkoxy silanes and/or condensation products thereof, and

a second composition (B) comprising
(B1) at least one compound selected from the group of amino acids and protein hydrolysates,

where
the total content of all polymers contained in the composition (A)—based on the total weight of the composition (A)—is below about 0.3% by weight, and
the total content of all polymers contained in the composition (B)—based on the total weight of the composition (B)—is below about 0.3% by weight.

If composition (A) was applied to the keratin material as part of a dyeing process, an increase in color intensity was observed, particularly if composition (B) was applied in the form of an after-treatment agent after application of composition (A) to the keratin material. In addition to the enhancement of color intensity, surprisingly, an improvement in wash fastness was also observed in this context. These effects could be obtained even without the presence of a polymer in compositions (A) and (B). When the dyeing procedure was repeated, no weakening of the color intensity was observed in the subsequent application.

### Treatment of Keratinous Material

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair.

Agents for treating keratinous material are understood to mean, for example, techniques for coloring the keratinous material, techniques for reshaping or shaping keratinous material, in particular keratinous fibers, or also techniques for conditioning or caring for the keratinous material. The agents prepared by the process of the present disclosure are particularly suitable for coloring keratinous material, in particular keratinous fibers, which are preferably human hair.

The term “coloring agent” is used in the context of the present disclosure to refer to a coloring of the keratin material, of the hair, caused using coloring compounds, such as thermochromic and photochromic dyes, pigments, mica, direct dyes and/or oxidation dyes. In this staining process, the colorant compounds are deposited in a particularly homogeneous and smooth film on the surface of the keratin material or diffuse into the keratin fiber. The film forms in situ by oligomerization or polymerization of the organic alkoxy silane(s), and by the interaction of the color-imparting compound and organic silicon compound and optionally other ingredients, such as a film-forming, polymer.

Organic C₁-C₆ Alkoxy Silanes (A1) and/or their Condensation Products in the Composition (A)

The composition (A) is exemplified in that it contains one or more organic C₁-C₆ alkoxy silanes (A1) and/or their condensation products.

The organic C₁-C₆ alkoxy silane(s) are organic, non-polymeric silicon compounds, preferably selected from the group of silanes containing one, two or three silicon atoms.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds of the present disclosure are preferably compounds containing one to three silicon atoms. Organic silicon compounds preferably contain one or two silicon atoms.

According to IUPAC rules, the term silane chemical compounds is based on a silicon skeleton and hydrogen. In organic silanes, the hydrogen atoms are completely or partially replaced by organic groups such as (substituted) alkyl groups and/or alkoxy groups.

A typical feature of the C₁-C₆ alkoxy silanes of the present disclosure is that at least one C₁-C₆ alkoxy group is directly bonded to a silicon atom. The C₁-C₆ alkoxy silanes according to the present disclosure thus comprise at least one structural unit R′R″R′″ Si—O—(C₁-C₆ alkyl) where the radicals R′, R″ and R′″ stand for the three remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolysable groups per molecule. If the hydrolysable C₁-C₆ alkoxy group is an ethoxy group, the organic silicon compound preferably contains a structural unit R′R″R′″Si—O—CH2—CH3. The R′, R″ and R′″ radicals again represent the three remaining free valences of the silicon atom.

Even the addition of insignificant amounts of water leads first to hydrolysis and then to a condensation reaction between the organic alkoxy silanes. For this reason, both the organic alkoxy silanes (A1) and their condensation products may be present in the composition.

A condensation product is understood to be a product formed by reaction of at least two organic C₁-C₆ alkoxy silanes with elimination of water and/or with elimination of a C₁-C₆ alkanol.

The condensation products can, for example, be dimers, or even trimers or oligomers, where in the condensation products are always in balance with the monomers.

Depending on the amount of water used or consumed in the hydrolysis, the equilibrium shifts from monomeric C₁-C₆ alkoxysilane to condensation product.

In a very particularly preferred embodiment, a process according to the present disclosure is exemplified in that the composition (A) comprises one or more organic C₁-C₆ alkoxy silanes (A1) selected from silanes having one, two or three silicon atoms, the organic silicon compound further comprising one or more basic chemical functions.

This basic group can be, for example, an amino group, an alkylamino group or a dialkylamino group, which is preferably connected to a silicon atom via a linker. Preferably, the basic group is an amino group, a C₁-C₆ alkylamino group or a di(C₁-C₆)alkylamino group.

A very particularly preferred method according to the present disclosure is exemplified in that the composition (A) comprises one or more organic C₁-C₆ alkoxy silanes (A1) selected from the group of silanes having one, two or three silicon atoms, and wherein the C₁-C₆ alkoxy silanes further comprise one or more basic chemical functions.

Particularly satisfactory results were obtained when C₁-C₆ alkoxy silanes of the formula (S-I) and/or (S-II) were used in the process according to the present disclosure. Since, as previously described, hydrolysis/condensation already starts at traces of moisture, the condensation products of the C₁-C₆ alkoxy silanes of formula (S-I) and/or (S-II) are also included in this embodiment.

In another very particularly preferred embodiment, a process according to the present disclosure is exemplified in that the first composition (A) comprises one or more organic C₁-C₆ alkoxy silanes (A1) of the formula (S-I) and/or (S-II),

R₁R₂N-L-Si(OR₃)ₐ(R₄)_{b} (S-I)

where

R₁, R₂ independently represent a hydrogen atom or a C₁-C₆ alkyl group,

L is a linear or branched divalent C₁-C₂₀ alkylene group,

R₃, R₄ independently of one another represent a C₁-C₆ alkyl group,

a, stands for an integer from 1 to 3, and

b stands for the integer 3a, and

(R₅O)_{c}(R₆)_{d}Si-(A)ₑ-[NR₇-(A′)]_{f}-[O-(A″)]_{g}-[NR₈-(A′″)]ₕ-Si(R₆′)_{d}′(OR₅′)_{c′} (S-II), where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (S-III),

(A″″)-Si(R₆″)_{d}″(OR₅″)_{c}″ (S-III),

c, stands for an integer from 1 to 3,

d stands for the integer 3-c,

c′ stands for an integer from 1 to 3,

d′ stands for the integer 3-c′,

c″ stands for an integer from 1 to 3,

d″ stands for the integer 3-c″,

e stands for 0 or 1,

f stands for 0 or 1,

g stands for 0 or 1,

h stands for 0 or 1,

provided that at least one of e, f, g and h is different from 0, and/or their condensation products.

The substituents R₁, R₂, R₃, R₄, R₅, R₅′, R₅″, R₆, R₆′, R₆″, R₇, R₈, L, A, A′, A″, A′″ and A″″ in the compounds of formula (S-I) and (S-II) are explained below as examples:
Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In the organic silicon compounds of the formula (S-I)

R₁R₂N-L-Si(OR₃)ₐ(R₄)_{b} (S-I),

the radicals R₁ and R₂ independently of one another represent a hydrogen atom or a C₁-C₆ alkyl group. Very preferably, R₁ and R₂ both represent a hydrogen atom.

In the middle part of the organic silicon compound is the structural unit or the linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each—L grouping may form—two bonds.

Preferably -L- stands for a linear, bivalent C₁-C₂₀ alkylene group. Further preferably -L- stands for a linear bivalent C₁-C₆ alkylene group. Particularly preferred—L stands for a methylene group (—CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—).

The organic silicon compounds of formula (S-I) according to the present disclosure.

R₁R₂N-L-Si(OR₃)ₐ(R₄)_{b} (S-I),

one end of each carries the silicon-containing group -Si(OR3)a(R4)b.

In the terminal structural unit —Si(OR₃)ₐ(R₄)_{b} R3 and R4 independently represent a C₁-C₆ alkyl group, and particularly preferably R₃ and R₄ independently represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Keratin treatment agents with particularly suitable properties could be prepared if the composition (A) contains at least one organic C₁-C₆ alkoxy silane of the formula (S-I) in which the radicals R₃, R₄ independently of one another represent a methyl group or an ethyl group.

Furthermore, dyeings with the best wash fastnesses could be obtained if the composition (A) contains at least one organic C₁-C₆ alkoxy silane of the formula (S-I) in which the radical a represents the number 3. In this case the radical b stands for the number 0.

In a further preferred embodiment, a process according to the present disclosure is exemplified in that the composition (A) comprises one or more organic C₁-C₆ alkoxy silanes of the formula (S-I),
where
R₃, R₄ independently of one another represent a methyl group or an ethyl group and
a stands for the number 3 and
b stands for the number 0.

In a further preferred embodiment, a process according to the present disclosure is exemplified in that the composition (A) comprises at least one or more organic C₁-C₆ alkoxy silanes of the formula (S-I),

R₁R₂N-L-Si(OR₃)ₐ(R₄)_{b} (S-I),

where

R₁, R₂ both represent a hydrogen atom, and

L represents a linear, bivalent C₁-C₆-alkylene group, preferably a propylene group (—CH₂—CH₂—CH₂—) or an ethylene group (—CH₂—CH₂—),

R₃ represents an ethyl group or a methyl group,

R₄ represents a methyl group or an ethyl group,

a stands for the number 3 and

b stands for the number 0.

Organic silicon compounds of the formula (I) which are particularly suitable for solving the problem according to the present disclosure are

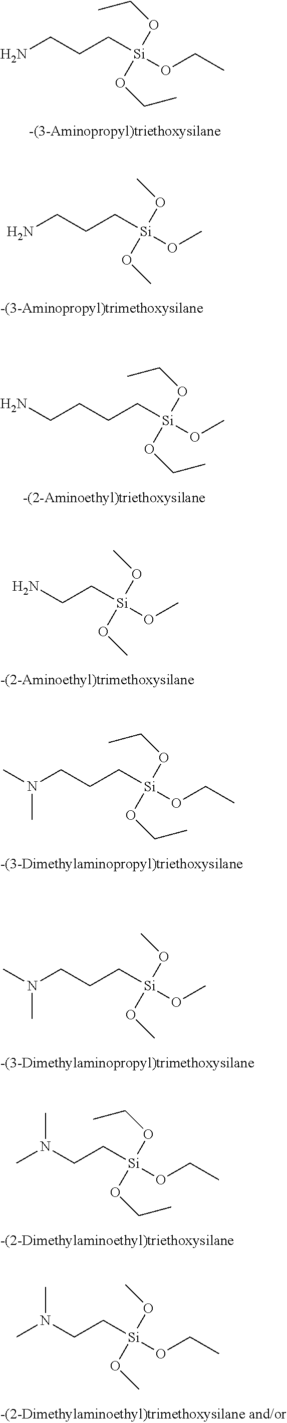

In a further preferred embodiment, a process according to the present disclosure is exemplified in that the first composition (A) comprises at least one organic C₁-C₆ alkoxysilane (A1) of formula (S-I) selected from the group of
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-Dimethylaminoethyl)triethoxysilane,
(2-Dimethylaminoethyl)trimethoxysilane
and/or their condensation products.

The organic silicon compound of formula (I) is commercially available. (3-aminopropyl)trimethoxysilane, for example, can be purchased from Sigma-Aldrich®. Also (3-aminopropyl)triethoxysilane is commercially available from Sigma-Aldrich®.

In a further embodiment of the process according to the present disclosure, composition (A) may also comprise one or more organic C₁-C₆ alkoxy silanes of formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II).

The organosilicon compounds of the formula (S-II) according to the present disclosure each carry at their two ends the silicon-containing groupings (R₅O)_{c}(R₆)_{d}Si— and —Si(R₆′)_{d′}(OR₅′)_{c′}.

In the central part of the molecule of formula (S-II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) according to the present disclosure contains at least one grouping from the group of -(A)- and —[NR₇-(A′)]- and —[O-(A″)]- and -[NR₈-(A′″)]-.

In the two terminal structural units (R₅O)_{c}(R₆)_{d}Si- and —Si(R₆′)_{d′}(OR₅′)_{c}′, the radicals R5, R5′, R5″ independently represent a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeings with the best wash fastness values could be obtained if the radicals c and c′ both stand for the number 3. In this case d and d′ both stand for the number 0.

In a further preferred embodiment, a process according to the present disclosure is exemplified in that the composition (A) comprises one or more organic C₁-C₆ alkoxy silanes of the formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),

where

R5 and R5′ independently represent a methyl group or an ethyl group,

c and c′ both stand for the number 3 and

d and d′ both stand for the number 0.

When c and c′ are both 3 and d and d′ are both 0, the organic silicon compounds according to the present disclosure correspond to the formula (S-IIa)

(R₅O)₃Si-(A)ₑ-[NR₇-(A′)]_{f}-[O-(A″)]_{g}-[NR₈-(A′″)]ₕ-Si(OR₅′)₃ (S-IIa).

The radicals e, f, g and h can independently stand for the number 0 or 1, whereby at least one radical from e, f, g and h is different from zero. The abbreviations e, f, g and h thus define which of the groupings -(A)ₑ- and —[NR₇-(A′)]_{f}- and —[O-(A″)]_{g}- and —[NR₈-(A′″)]ₕ- are in the middle part of the organic silicon compound of formula (II).

In this context, the presence of certain groupings has proven to be particularly advantageous in terms of achieving washfast dyeing results. Particularly satisfactory results could be obtained if at least two of the radicals e, f, g and h stand for the number 1. Especially preferred e and f both stand for the number 1. Furthermore, g and h both stand for the number 0.

When e and f are both 1 and g and h are both 0, the organic silicon compounds according to the present disclosure are represented by the formula (S-IIb)

(R₅O)_{c}(R₆)_{d}Si-(A)-[NR₇-(A′)]-Si(R₆′)_{d′}(OR₅′)_{c′} (S-IIb).

The radicals A, A′, A″, A′″ and A″″ independently represent a linear or divalent, bivalent C₁-C₂₀ alkylene group. Preferably the radicals A, A′, A″, A′″ and A″″ independently of one another represent a linear, bivalent C₁-C₂₀ alkylene group. Further preferably the radicals A, A′, A″, A′″ and A″″ independently represent a linear bivalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). Very preferably, the radicals A, A′, A″, A′″ and A″″ represent a propylene group (—CH₂—CH₂—CH₂—).

If the radical f represents the number 1, then the organic silicon compound of formula (II) according to the present disclosure contains a structural grouping —[NR₇-(A′)]-. If the radical h represents the number 1, then the organic silicon compound of formula (II) according to the present disclosure contains a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (S-III)

-(A″″)-Si(R₆″)_{d}″(OR₅″)_{c}″ (S-III).

Very preferably the radicals R7 and R8 independently of one another represent a hydrogen atom, a methyl group, a 2-hydroxyethyl group, a 2-alkenyl group, a 2-aminoethyl group or a grouping of the formula (S-III).

If the radical f represents the number 1 and the radical h represents the number 0, the organic silicon compound according to the present disclosure contains the grouping [NR₇-(A′)] but not the grouping —[NR₈-(A′″)]. If the radical R7 now stands for a grouping of the formula (III), the organic silicone compound comprises 3 reactive silane groups.

In a further preferred embodiment, a process according to the present disclosure is exemplified in that the composition (A) comprises one or more organic C₁-C₆ alkoxy silanes (A1) of the formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where

e and f both stand for the number 1,

g and h both stand for the number 0,

A and A′ independently represent a linear, divalent C₁-C₆ alkylene group and

R7 represents a hydrogen atom, a methyl group, a 2-hydroxyethyl group, a 2-alkenyl group, a 2-aminoethyl group or a group of formula (S-III).

In a further preferred embodiment, a process according to the present disclosure is exemplified in that the composition (A) comprises one or more organic C₁-C₆ alkoxy silanes (A1) of the formula (S-II), where

e and f both stand for the number 1,

g and h both stand for the number 0,

A and A′ independently of one another represent a methylene group(—CH₂—), an ethylene group (—CH₂—CH₂—) or a propylene group (—CH₂—CH₂—CH₂), and

R7 represents a hydrogen atom, a methyl group, a 2-hydroxyethyl group, a 2-alkenyl group, a 2-aminoethyl group or a group of formula (S-III).

Organic silicon compounds of the formula (S-II) which are well suited for solving the problem according to the present disclosure are

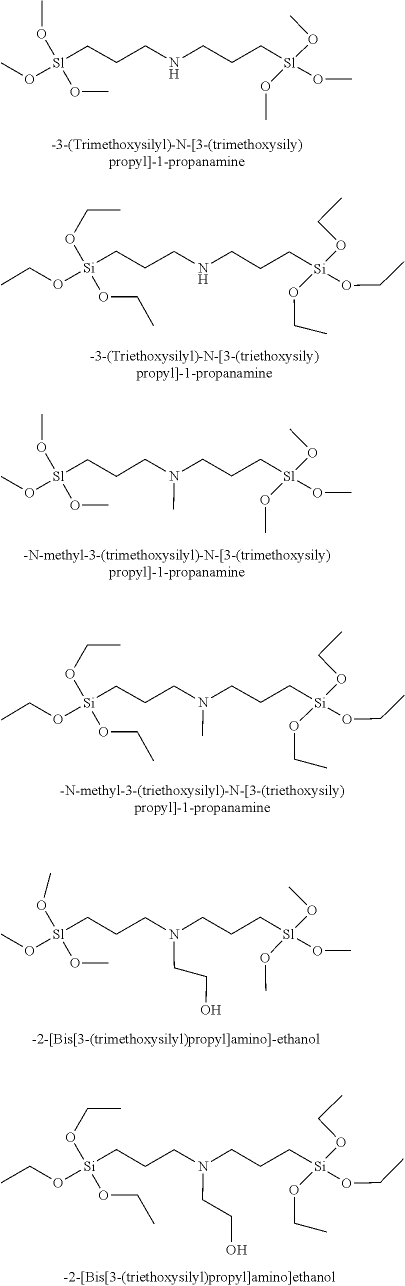

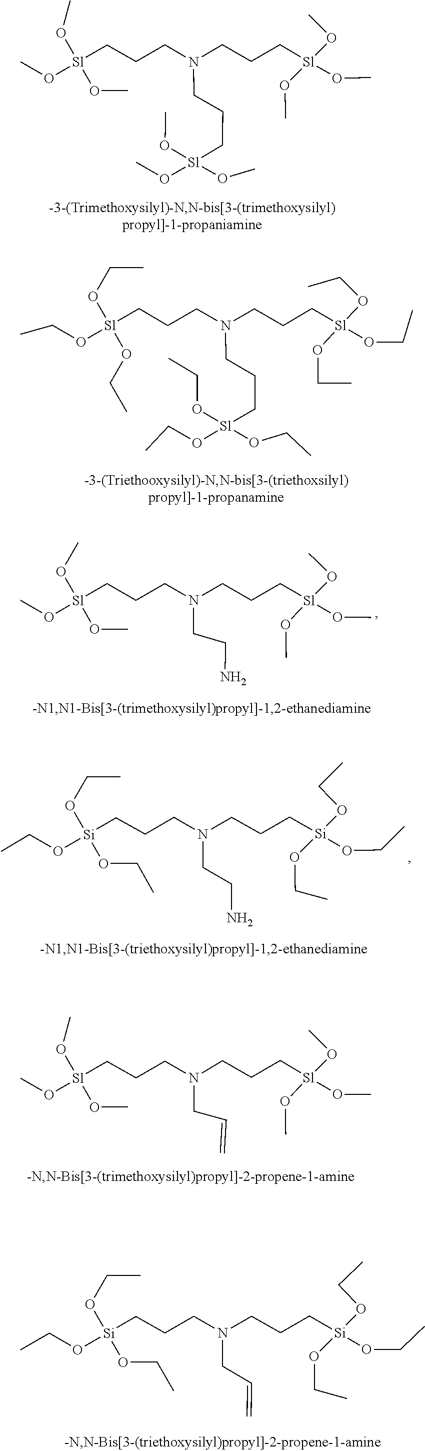

The organic silicon compounds of formula (S-II) are commercially available. Bis(trimethoxysilylpropyl)amines with the CAS number 82985-35-1 can be purchased from Sigma-Aldrich®.
Bis[3-(triethoxysilyl)propyl]amines with the CAS number 13497-18-2 can be purchased from Sigma-Aldrich®, for example.
N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich® or Fluorochem®.
3-(triethoxysilyl)-N,N-bis[3-(triethoxysilyl)propyl]-1-propanamine with the CAS number 18784-74-2 can be purchased for example from Fluorochem® or Sigma-Aldrich®.

In a further preferred embodiment, a process according to the present disclosure is exemplified in that the composition (A) comprises one or more organic C₁-C₆ alkoxy silanes of formula (S-II) selected from the group of
3-(Trimethoxysilyl)-N[3-(trimethoxysilyl)propyl]-1-propanamine
3-(Triethoxysilyl)-N-[3-(triethoxysilyl) propyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[Bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-Bis[3-(trimethoxysilyl) propyl]-1,2-ethanediamine,
N1,N1-Bis[3-(triethoxysilyl) propyl]-1,2-ethanediamine,
N,N-Bis[3-(trimethoxysilyl)propyl]-2-Propen-1-amine and/or
N,N-Bis[3-(triethoxysilyl)propyl]-2-propen-1-amine,
and/or their condensation products.

In further dyeing trials, it has also been found to be particularly advantageous if at least one organic C₁-C₆ alkoxy silane (A1) of the formula (S-IV) was used in the process according to the present disclosure

R₉Si(OR₁₀)ₖ(R₁₁)ₘ (S-IV).

The compounds of formula (S-IV) are organic silicon compounds selected from silanes having one, two or three silicon atoms, wherein the organic silicon compound comprises one or more hydrolysable groups per molecule.

The organic silicon compound(s) of formula (S-IV) may also be referred to as silanes of the alkyl-C₁-C₆-alkoxy-silane type,

R₉Si(OR₁₀)ₖ(R₁₁)ₘ (S-IV),

where

R₉ represents a C₁-C₁₂ alkyl group,

R₁₀ represents a C₁-C₆ alkyl group,

R₁₁ represents a C₁-C₆ alkyl group

k is an integer from 1 to 3, and

m stands for the integer 3-k.

In a further embodiment, a particularly preferred method according to the present disclosure is exemplified in that the first composition (A) contains one or more organic C₁-C₆ alkoxy silanes (A1) of the formula (S-IV),

R₉Si(OR₁₀)ₖ(R₁₁)ₘ (S-IV),

where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k,
and/or their condensation products.

In the organic C₁-C₆ alkoxy silanes of formula (S-IV), the R₉ radical represents a C₁-C₁₂ alkyl group. This C₁-C₁₂ alkyl group is saturated and can be linear or branched. Preferably, R₉ represents a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R9 stands for a methyl group, an ethyl group or an n-octyl group.

In the organic silicon compounds of formula (S-IV), the radical R₁₀ represents a C₁-C₆ alkyl group. Highly preferred R₁₀ stands for a methyl group or an ethyl group.

In the organic silicon compounds of formula (S-IV), the radical R₁₁ represents a C₁-C₆ alkyl group. Particularly preferably, R₁₁ represents a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Dyeings with the best wash fastnesses could be obtained when the composition (A) contains at least one organic C₁-C₆ alkoxy silane (A1) of formula (S-IV) in which the radical k represents the number 3. In this case the radical m stands for the number 0.

Organic silicon compounds of the formula (S-IV) which are particularly suitable for solving the problem according to the present disclosure are

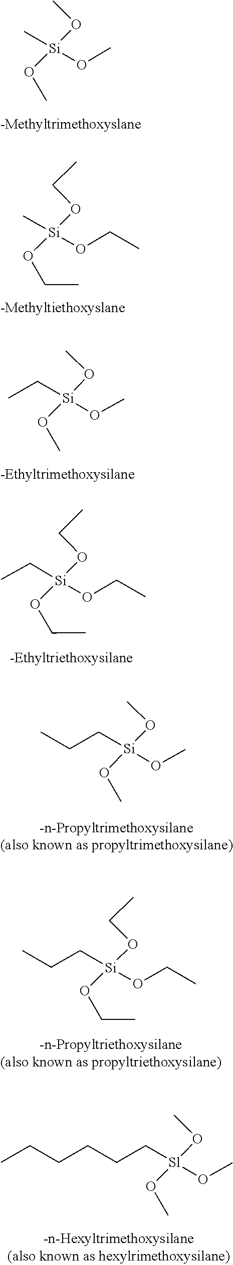

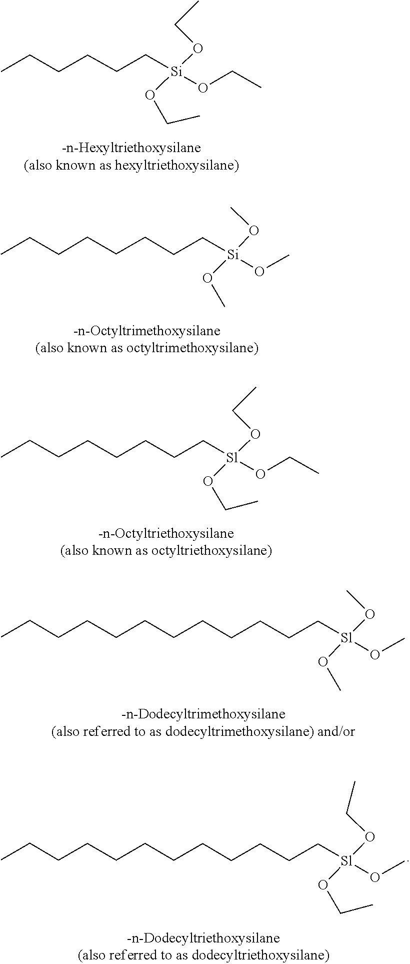

In a further preferred embodiment, a process according to the present disclosure is exemplified in that the first composition (A) comprises at least one organic C₁-C₆ alkoxysilane (A1) of formula (S-IV) selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
and/or their condensation products.

The corresponding hydrolysis or condensation products are, for example, the following. Here, the condensation products represent maximally oligomeric compounds, but not polymers.

Hydrolysis of C₁-C₆ alkoxy silane of the formula (S-I) with water (reaction scheme using the example of 3-aminopropyltriethoxysilane):

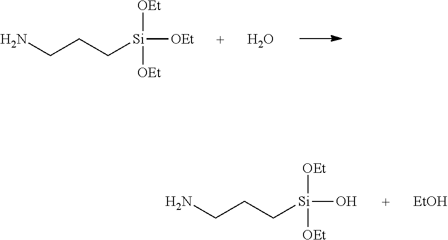 [SMILES: "EtOH" ; CCO[Si](CCCN)(OCC)OCC ; O ; CCO[Si](O)(CCCN)OCC]

Depending on the amount of water used, the hydrolysis reaction can also take place several times per C₁-C₆ alkoxy silane used:

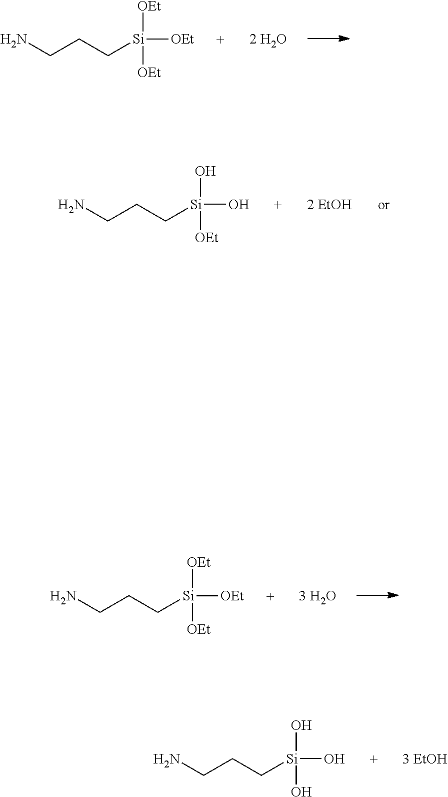

Hydrolysis of C₁-C₆ alkoxy silane of formula (S-IV) with water (reaction scheme using methyltrimethoxysilane as an example):

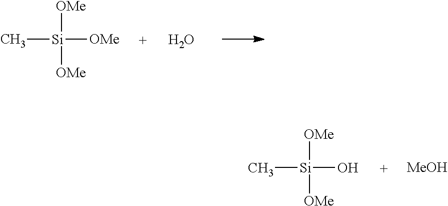

Depending on the amount of water used, the hydrolysis reaction can also take place several times per C₁-C₆ alkoxy silane used:

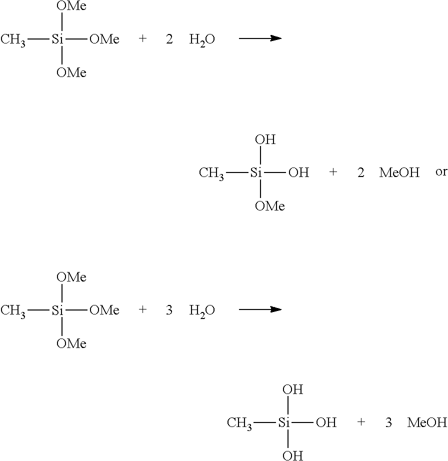

Condensation reactions include (shown using the mixture (3-aminopropyl)triethoxysilane and methyltrimethoxysilane):

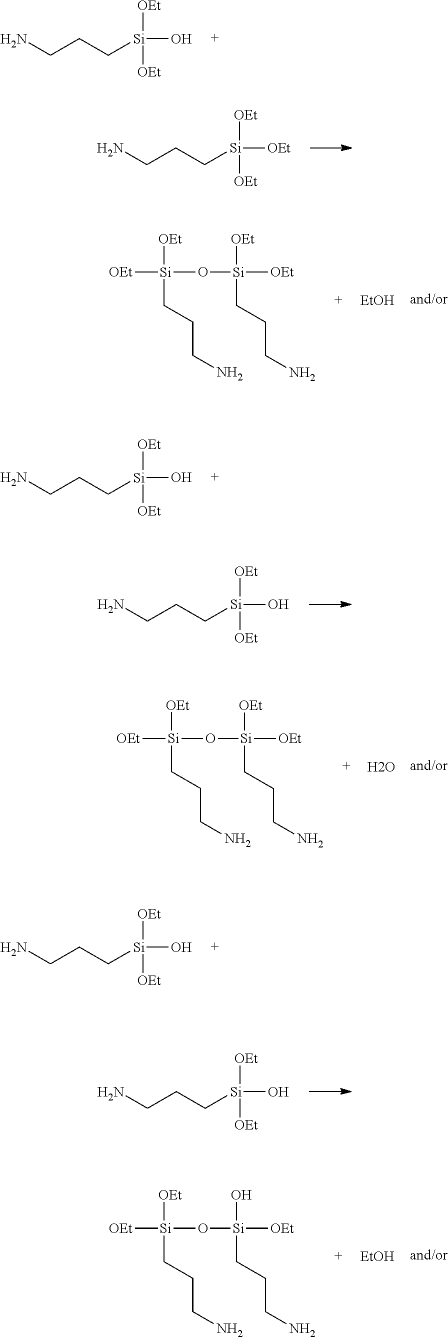

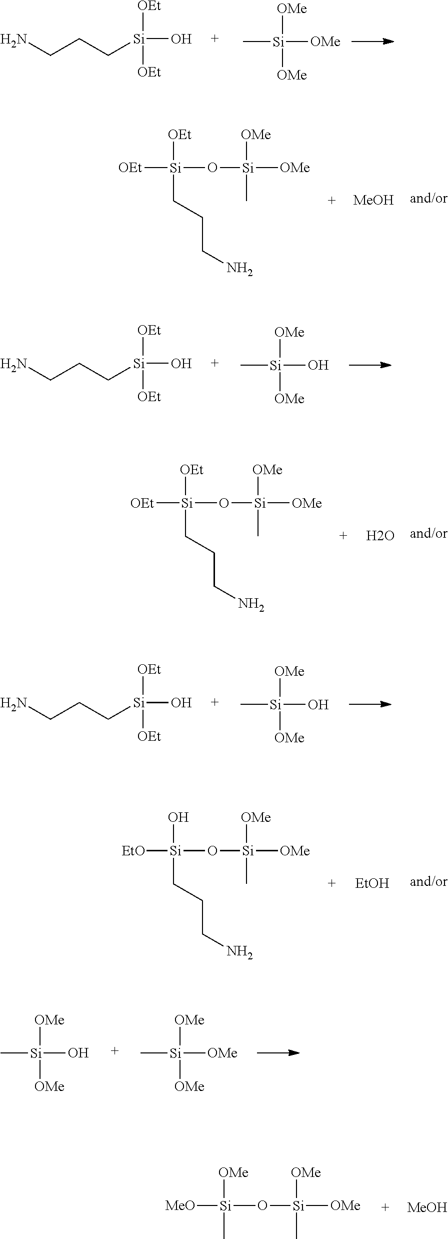

In the above exemplary reaction schemes the condensation to a dimer is shown in each case, but further condensations to oligomers with several silane atoms are also possible and preferred.

Both partially hydrolyzed and fully hydrolyzed C₁-C₆ alkoxysilanes of the formula (S-I) can participate in these condensation reactions, which undergo condensation with yet unreacted, partially or also fully hydrolyzed C₁-C₆ alkoxysilanes of the formula (S-I). In this case, the C₁-C₆ alkoxysilanes of formula (S-I) react with themselves.

Furthermore, both partially hydrolyzed and fully hydrolyzed C₁-C₆-alkoxysilanes of the formula (S-I) can also participate in the condensation reactions, which undergo condensation with not yet reacted, partially or also fully hydrolyzed C₁-C₆-alkoxysilanes of the formula (S-IV). In this case, the C₁-C₆ alkoxysilanes of formula (S-I) react with the C₁-C₆ alkoxysilanes of formula (S-IV).

Furthermore, both partially hydrolyzed and fully hydrolyzed C₁-C₆-alkoxysilanes of the formula (S-IV) can also participate in the condensation reactions, which undergo condensation with not yet reacted, partially or also fully hydrolyzed C₁-C₆-alkoxysilanes of the formula (S-IV). In this case, the C₁-C₆ alkoxysilanes of formula (S-IV) react with themselves.

The composition (A) according to the present disclosure may contain one or more organic C₁-C₆ alkoxysilanes (A1) in various proportions. The skilled person determines this depending on the desired thickness of the silane coating on the keratin material and on the amount of keratin material to be treated.

Particularly storage-stable preparations with very good dyeing results in application could be obtained when the composition (A) contains—based on its total weight—one or more organic C₁-C₆-alkoxysilanes (A1) and/or the condensation products thereof in a total amount of from about 30.0 to about 85.0% by weight, preferably from about 35.0 to about 80.0% by weight, more preferably from about 40.0 to about 75.0% by weight, still more preferably from about 45.0 to about 70.0% by weight, and most preferably from about 50.0 to about 65.0% by weight.

In a further embodiment, a very particularly preferred process is exemplified in that the first composition (A) comprises—based on the total weight of the composition (A)—one or more organic C₁-C₆-alkoxysilanes (A2) and/or the condensation products thereof in a total amount of from about 30.0 to about 85.0 wt.- %, preferably from about 35.0 to about 80.0% by weight, more preferably from about 40.0 to about 75.0% by weight, still more preferably from about 45.0 to about 70.0% by weight and most preferably from about 50.0 to about 65.0% by weight.

### Other Cosmetic Ingredients in the Composition (A)

In addition, the composition (A) may also contain one or more other cosmetic ingredients.

The cosmetic ingredients that may be optionally used in the composition (A) may be any suitable ingredients to impart further beneficial properties to the product. For example, in the composition (A), a solvent, a surface-active compound from the group of nonionic, cationic, anionic or zwitterionic/amphoteric surfactants, coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

As previously described, the content of polymers in the composition (A) is limited to a maximum of about 0.3% by weight, or preferably excluded entirely.

The selection of these other substances will be made by the specialist according to the desired properties of the agents. Regarding other optional components and the quantities of these components used, explicit reference is made to the relevant manuals known to the specialist.

In this context, it has proved particularly preferred to use in composition (A) a cosmetic ingredient selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane.

In another particularly preferred embodiment, a process according to the present disclosure is exemplified in that the first composition (A) comprises at least one cosmetic ingredient selected from the group of hexamethyldisiloxane. contains octamethyltrisiloxane, decamethyltetrasiloxane, hexamethylcyclotrisiloxane, octamethylcyclotetrasiloxane and decamethylcyclopentasiloxane.
Hexamethyldisiloxane has the CAS number 107-46-0 and can be purchased commercially from Sigma-Aldrich®, for example.

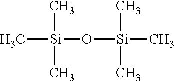

Octamethyltrisiloxane has the CAS number 107-51-7 and is also commercially available from Sigma-Aldrich®.

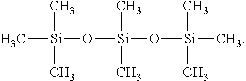

Decamethyltetrasiloxane carries the CAS number 141-62-8 and is also commercially available from Sigma-Aldrich®.

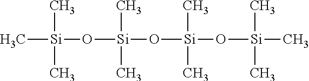

Hexamethylcyclotrisiloxane has the CAS No. 541-05-9.
Octamethylcyclotetrasiloxane has the CAS No. 556-67-2.
Decamethylcyclopentasiloxane has the CAS No. 541-02-6.

The use of hexamethyldisiloxane in composition (A) has proved to be particularly preferred. Particularly preferably, hexamethyldisiloxane is present—based on the total weight of composition (A)—in amounts of from about 1.0 to about 20.0% by weight, preferably from about 1.3 to about 10.0% by weight, further preferably from about 1.6 to about 5.0% by weight and very particularly preferably from about 2.0 to about 4.0% by weight in composition (A).

### Water Content (A1) in the Composition (A)

The process according to the present disclosure is exemplified by the application of a first composition (A) on the keratinous material.

In the context of the present disclosure, composition (A) is a ready-to-use composition which, in its present embodiment, can be applied to the keratin material in particular to the hair.

In the process according to the present disclosure, the composition (A) can either be provided in its present form in a container. However, with the C₁-C₆ alkoxy silanes, the composition (A) contains very reactive compounds. However, to avoid problems related to storage stability, it is particularly preferred to prepare the ready-to-use and reactive composition (A) just before use by mixing two or more storage-stable compositions. For example, the ready-to-use composition (A) can be prepared by mixing a low-water silane blend (A-I), which contains the organic C₁-C₆ alkoxy silane(s) (A1) in concentrated form, and a water-rich carrier formulation (A-II), which can be, for example, a gel, a lotion or a surfactant system.

Accordingly, the ready-to-use composition (A) preferably has a higher water content, which—based on the total weight of the composition (A)—may be in the range from about 50.0 to about 90.0% by weight, preferably from about 55.0 to about 90.0% by weight, further preferably from about 60.0 to about 90.0% by weight and particularly preferably from about 70.0 to about 90.0% by weight.

In the context of a further embodiment, a process according to the present disclosure is exemplified in that the first composition (A) contains—based on the total weight of the composition (A)—from about 50.0 to about 90.0% by weight, preferably from about 55.0 to about 90.0% by weight, further preferably from about 60.0 to about 90.0% by weight and particularly preferably from about 70.0 to about 90.0% by weight of water.

### pH Value of the Compositions (A)

In further experiments it has been found that the pH values of composition (A) can have an influence on the color intensities obtained during dyeing. It was found that alkaline pH values have a beneficial effect on the dyeing performance achievable in the process.

For this reason, it is preferred that the compositions (A) have a pH of from about 7.0 to about 12.0, preferably from about 7.5 to about 11.5, more preferably from about 8.0 to about 11.0, and most preferably from about 8.0 to about 10.5.

The pH value can be measured using the usual methods known from the state of the art, such as pH measurement using glass electrodes via combination electrodes or using pH indicator paper.

In another very particularly preferred embodiment, a process according to the present disclosure is exemplified in that the composition (A) has a pH of from about 7.0 to about 12.0, preferably from about 7.5 to about 11.5, more preferably from about 8.0 to about 11.0 and most preferably from about 8.0 to about 10.5.

To adjust the above pH values, the alkalizing agents can be used, which can also be used to adjust the pH value of composition (B).

Amino Acids, Protein Hydrolysates and/or Proteins in the Composition (B).

The method according to the present disclosure comprises the application of a second composition (B) on the keratin material. Here, the composition (B) is exemplified in that it contains at least one compound selected from the group of amino acids and/or protein hydrolysates.

An amino acid is a chemical compound with an amino group and a carboxylic acid group. The class of amino acids includes organic compounds containing at least one amino group (—NH₂ or substituted —NR₂) and a carboxy group (—COOH) as functional groups, i.e. have structural components of the amines and carboxylic acids. Chemically, they can be distinguished according to the position of their amino group to the carboxy group—if the amino group at the C_{α}-atom is directly adjacent to the terminal carboxy group, this is called α-constant and speaks of α amino acids. Carboxylic acids with a total number of C atoms of C2-20 are preferred, more preferably of C2-15, especially preferably of C2-10.

Preferred amino acids are selected from arginine, lysine, histidine, asparagine, glutamine, cysteine, methionine, tryptophan, serine, alanine, aspartic acid, glutamic acid, glycine, isoleucine, leucine, phenylalanine, proline, threonine, tyrosine and valine, and mixtures of these amino acids.

Chiral amino acids have a stereogenic center and can occur in mirror-image forms. For example, arginine occurs in the form of L-arginine and D-arginine. Both the L-form of an amino acid and its D-form, as well as mixtures thereof, are encompassed by the present disclosure. Accordingly, within the scope of the present disclosure, both enantiomers can be used equally as specific compounds or also mixtures thereof, as racemates. However, it is particularly advantageous to use the naturally preferred isomeric form, usually in L-configuration.

In another particularly preferred embodiment, a process according to the present disclosure is exemplified in that the second composition (B) comprises at least one amino acid selected from the group of arginine, lysine, histidine, asparagine, glutamine, cysteine, methionine, tryptophan, serine, alanine, aspartic acid, glutamic acid, glycine, isoleucine, leucine, phenylalanine, proline, threonine, tyrosine and valine.

The best results were obtained with arginine.

In the context of a further particularly preferred embodiment, a method according to the present disclosure is exemplified in that the second composition (B) comprises arginine.

To achieve the best possible wash fastnesses, the amino acid(s) in composition (B) are preferably used in specific ranges of amounts. It has been found to be particularly advantageous if the composition (B) contains one or more amino acids in a total amount of about 0.1 to about 20.0% by weight, preferably about 0.5 to about 10.0% by weight, based on the total weight of the composition (B).

In the context of a further particularly preferred embodiment, a process according to the present disclosure is exemplified in that the second composition (B) contains—based on the total weight of the composition (B)—one or more amino acids in a total amount of about 0.1 to about 20.0% by weight, preferably about 0.5 to about 10.0% by weight.

Further good dyeing results with high color intensity and additionally improved wash fastness could be achieved if in the composition (B) at least one protein hydrolysate was used in addition to or instead of the amino acid.

According to the present disclosure, protein hydrolysates are degradation products of proteins, which are produced by acidic, basic or enzymatic reaction. Due to the manufacturing process, protein hydrolysates exhibit a distribution of molecular weight. The protein hydrolysates according to the present disclosure also include oligopeptides since these can also be produced from proteins by appropriate reactions. According to the present disclosure, individual amino acids, which are present as discrete individual compounds, do not count as protein hydrolysates within the meaning of the present disclosure. According to the present disclosure, protein hydrolysates of both plant and animal or marine or synthetic origin can be used.

Protein hydrolysates within the meaning of the present disclosure are understood to be at most oligomeric compounds composed of a maximum of about 10 amino acids.

Animal protein hydrolysates include elastin, collagen, keratin, silk and milk protein hydrolysates, which may also be in the form of salts. Such products are marketed under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex®), ProSina® (Croda®) and Kerasol® (Croda®).

Furthermore, vegetable protein hydrolysates preferred according to the present disclosure include soy, almond, pea, moringa, potato and wheat protein hydrolysates. Such products are available, for example, under the trademarks Gluadin® (Cognis), DiaMin® (Diamalt), Lexein® (Inolex®), Hydrosoy® (Croda®), Hydrolupin® (Croda®), Hydrosesame® (Croda®), Hydrotritium® (Croda®), Crotein® (Croda®) and Puricare® LS 9658 from Laboratoires Sérobiologiques.

Other protein hydrolysates preferred according to the present disclosure are of marine origin. These include, for example, collagen hydrolysates from fish or algae as well as protein hydrolysates from mussels or pearl hydrolysates. Examples of pearl extracts according to the present disclosure are the commercial products Pearl Protein Extract BG® or Crodarom® Pearl.

Furthermore, cationized protein hydrolysates are to be counted among the protein hydrolysates, whereby the underlying protein hydrolysate can originate from animals, for example from collagen, milk or keratin, from plants, for example from wheat, corn, rice, potatoes, soy or almonds, from marine life forms, for example from fish collagen or algae, or biotechnologically obtained protein hydrolysates. Typical examples of the cationic protein hydrolysates and derivatives of the present disclosure are the products listed under the INCI designations in the “International Cosmetic Ingredient Dictionary and Handbook” (seventh edition 1997, The Cosmetic, Toiletry, and Fragrance Association 1101 17^{th} Street, N.W., Suite 300, Washington, D.C. 20036-4702) and commercially available.

Satisfactory results were observed when composition (B) contained at least one protein hydrolysate selected from the hydrolysates of elastin, collagen, keratin, silk, milk protein, and soy, almond, pea, moringa, potato and wheat protein hydrolysates.

In another particularly preferred embodiment, a method according to the present disclosure is exemplified in that the second composition (B) comprises a protein hydrolysate selected from the group of protein hydrolysates of elastin, collagen, keratin, silk, milk protein, soy, almond, pea, moringa, potato and wheat protein hydrolysates.

To achieve the best possible wash fastness properties, the protein hydrolysate(s) in composition (B) are preferably used in certain quantity ranges. It has been found to be particularly advantageous if the composition (B) contains—based on the total weight of the composition (B)—one or more protein hydrolysates in a total amount of about 0.1 to about 20.0% by weight, preferably about 0.5 to about 10.0% by weight.

In the context of a further particularly preferred embodiment, a process according to the present disclosure is exemplified in that the second composition (B) contains—based on the total weight of the composition (B)—one or more protein hydrolysates in a total amount of about 0.1 to about 20.0% by weight, preferably about 0.5 to about 10.0% by weight.

Also, protein hydrolysates according to the present disclosure are oligopeptides. Oligopeptides may be preferred in the hair treatment compositions of the present disclosure due to their defined amino acid sequence.

An oligopeptide comprising at least one amino acid sequence Glu-Glu-Glu

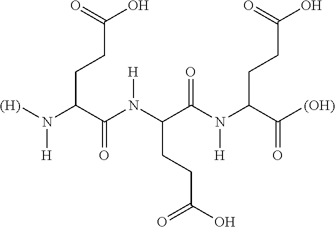

wherein the amino group may be free or protonated and the carboxy groups may be free or deprotonated, may be particularly preferred according to the present disclosure. In this formula as in all the formulas below, the bracketed hydrogen atom of the amino group as well as the bracketed hydroxy group of the acid function means that the groups in question may be present as such (in which case it is an oligopeptide with the relevant number of amino acids as in the formula above, or else that the amino acid sequence is present in an oligopeptide comprising further amino acids—depending on where the further amino acid(s) is/are bonded, the bracketed components of the above formula are replaced by the further amino acid residue(s).

Oligopeptides within the meaning of the present application are condensation products of amino acids linked by peptide bonds in the manner of acid amides, comprising at least 3 and at most about 25 amino acids. In hair treatment compositions preferred according to the present disclosure, the oligopeptide comprises 5 to about 15 amino acids, preferably 6 to about 13 amino acids, particularly preferably 7 to about 12 amino acids and especially 8, 9 or 10 amino acids. Depending on whether further amino acids are bound to the Glu-Glu-Glu sequence and depending on the nature of these amino acids, the molar mass of the oligopeptide contained in the agents of the present disclosure may vary. Hair treatment compositions preferred according to the present disclosure are exemplified in that the oligopeptide has a molecular weight of from about 650 to about 3000 Da, preferably from about 750 to about 2500 Da, particularly preferably from about 850 to about 2000 Da and especially from about 1000 to about 1600 Da. As can be seen from the preferred number of amino acids in the oligopeptides and the preferred molecular weight range, oligopeptides are preferably used that do not solely include the three glutamic acids but have other amino acids bound to this sequence. These further amino acids are preferably selected from certain amino acids, while certain other representatives are less preferred according to the present disclosure. A particularly preferred oligopeptide additionally contains tyrosine, which is preferably linked to the Glu-Glu-Glu sequence via its acid function. Hair treatment compositions preferred according to the present disclosure are therefore exemplified in that the oligopeptide contained therein comprises at least one amino acid sequence Tyr-Glu-Glu-Glu

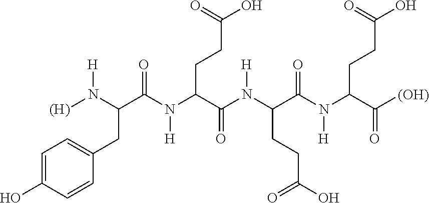

wherein the amino group may be free or protonated and the carboxy groups may be free or deprotonated.

Another particularly preferred oligopeptide additionally contains isoleucine, which is preferably linked to the Glu-Glu-Glu sequence via its amino function. Hair treatment compositions preferred according to the present disclosure are therefore exemplified in that the oligopeptide contained therein comprises at least one amino acid sequence Glu-Glu-Glu-Ile

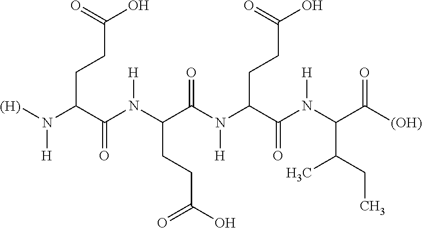

wherein the amino group may be free or protonated and the carboxy groups may be free or deprotonated.

Oligopeptides comprising both amino acids (tyrosine and isoleucine) are preferred according to the present disclosure. Particularly preferred are hair treatment compositions according to the present disclosure in which the oligopeptide contained therein comprises at least one amino acid sequence Tyr-Glu-Glu-Ile

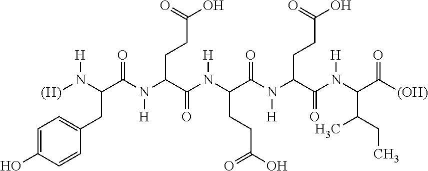

wherein the amino group may be free or protonated and the carboxy groups may be free or deprotonated.

Further preferred oligopeptides additionally contain arginine, which is preferably bound to isoleucine.

Even further preferred oligopeptides additionally contain valine, which is preferably present bound to the arginine. Hair treatment compositions further preferred according to the present disclosure are therefore exemplified in that the oligopeptide contained therein comprises at least one amino acid sequence Tyr-Glu-Glu-Ile-Arg-Val

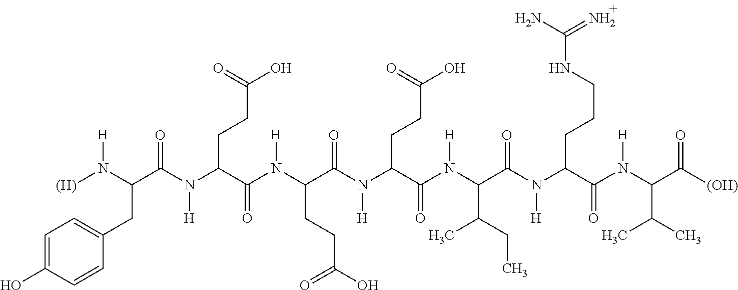

wherein the amino groups may be free or protonated and the carboxy groups may be free or deprotonated.

Even more preferred oligopeptides additionally contain leucine, which is preferably present bound to valine. Hair treatment compositions further preferred according to the present disclosure are exemplified in that the oligopeptide contained therein comprises at least one amino acid sequence Tyr-Glu-Glu-Ile-Arg-Val-Leu

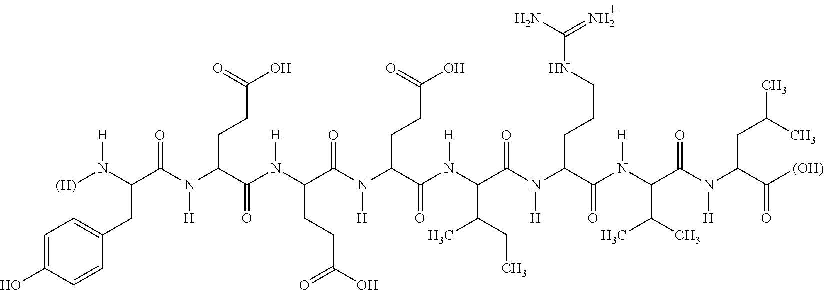

wherein the amino groups may be free or protonated and the carboxy groups may be free or deprotonated.

Particularly preferred oligopeptides additionally contain leucine, which is preferably present bound to the tyrosine. Hair treatment compositions further preferred according to the present disclosure are exemplified in that the oligopeptide contained therein comprises at least one amino acid seauence Leu-Tyr-Glu-Glu-Ile-Ar-Val-Leu

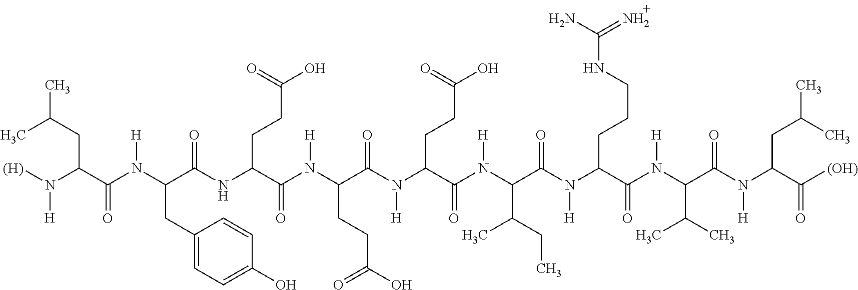

wherein the amino groups may be free or protonated and the carboxy groups may be free or deprotonated.

### Water Content of the Composition (B)

Composition (B) contains the amino acid(s), protein hydrolysates and/or proteins in a cosmetic carrier, preferably in an aqueous cosmetic carrier.

In this context, it has been found to be preferred if the composition (B) contains—based on the total weight of the composition (B)—about 5.0 to about 99.0% by weight, preferably about 15.0 to about 97.0% by weight, more preferably about 25.0 to about 97.0% by weight, still more preferably about 35.0 to about 97.0% by weight and very particularly preferably about 45.0 to about 97.0% by weight of water.

In a further embodiment, a process according to the present disclosure is exemplified in that the second composition (B) contains—based on the total weight of the composition (B)—from about 5.0 to about 99.0% by weight, preferably from about 15.0 to about 97.0% by weight, more preferably from about 25.0 to about 97.0% by weight, still more preferably from about 35.0 to about 97.0% by weight and very particularly preferably from about 45.0 to about 97.0% by weight of water.

### Other Cosmetic Ingredients in the Composition (B)

In addition, the composition (B) may also contain one or more further cosmetic ingredients.

The cosmetic ingredients that may be optionally used in the composition (B) may be any suitable ingredients to impart further beneficial properties to the product. For example, in the composition (B), a solvent, a surface-active compound from the group of nonionic, cationic, anionic or zwitterionic/amphoteric surfactants, coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives and plant extracts.

As previously described, the content of polymers in the composition (B) is limited to a maximum of about 0.3% by weight, or preferably excluded entirely.

The selection of these other substances will be made by the specialist according to the desired properties of the agents. Regarding other optional components and the quantities of these components used, explicit reference is made to the relevant manuals known to the specialist.

### pH Value of the Compositions (B)

In further tests, it has been found that the pH values of composition (B) can also have an influence on the color intensities and wash fastnesses obtained during dyeing. It was found that alkaline pH values have a beneficial effect on the dyeing performance achievable in the process.

For this reason, it is preferred that the compositions (B) have a pH of from about 7.0 to about 12.0, preferably from about 7.5 to about 11.5, more preferably from about 8.0 to about 11.0, and most preferably from about 8.0 to about 10.5.

The pH value can be measured using the usual methods known from the state of the art, such as pH measurement using glass electrodes via combination electrodes or using pH indicator paper.

In another very particularly preferred embodiment, a process according to the present disclosure is exemplified in that the composition (B) has a pH of from about 7.0 to about 12.0, preferably from about 7.5 to about 11.5, more preferably from about 8.0 to about 11.0, and most preferably from about 8.0 to about 10.5.

To adjust this alkaline pH, it may be necessary to add an alkalizing agent and/or acidifying agent to the reaction mixture. The pH values for the purposes of the present disclosure are pH values measured at a temperature of 22° C.

For example, ammonia, alkanolamines and/or basic amino acids can be used as alkalizing agents.

Alkanolamines may be selected from primary amines having a C₂-C₆ alkyl parent bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3 -amino-2-methylpropan-1-ol, 1-amino-2-methylpropan-2-ol, 3-aminopropan-1,2-diol, 2-amino-2-methylpropan-1,3-diol.

If basic amino acids are used in composition (B), it is also possible to adjust the pH value by adding the basic amino acids themselves. According to the present disclosure, basic amino acids are those amino acids which have an isoelectric point pI of greater than about 7.0.

Basic α-aminocarboxylic acids contain at least one asymmetric carbon atom. In the context of the present disclosure, both enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, an agent according to the present disclosure is therefore exemplified in that the alkalizing agent is a basic amino acid from the group arginine, lysine, ornithine and/or histidine.

In addition, inorganic alkalizing agents can also be used. Inorganic alkalizing agents usable according to the present disclosure are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Particularly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-Amino-2-methylpropan-2-ol, 3-aminopropan-1,2-diol, 2-amino-2-methylpropan-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Apart from the alkalizing agents described above, experts are familiar with common acidifying agents for fine adjustment of the pH value. According to the present disclosure, preferred acidifiers are pleasure acids, such as citric acid, acetic acid, malic acid or tartaric acid, as well as diluted mineral acids.

### No use of Polymers in Composition (A) and (B)

The process according to the present disclosure is exemplified in that the compositions (A) and (B) used in the process are free of polymers.

It is typical of the composition (A) that the total content of all polymers contained in the composition (A)—based on the total weight of the composition (A)—is below about 0.3% by weight.

Composition (B) is also exemplified in that the total content of all polymers contained in composition (B)—based on the total weight of composition (B)—is below about 0.3% by weight.

By omitting polymers in both compositions (A) and (B), a way was found to subject keratinous material, especially keratinous fibers such as hair, to repeated dyeing processes without the user noticing a reduction in color intensity during subsequent applications.

Polymers are macromolecules with a molecular weight of at least about 1000 g/mol, preferably of at least about 2500 g/mol, particularly preferably of at least about 5000 g/mol, which include identical, repeating organic units. The polymers of the present disclosure may be synthetically produced polymers which are manufactured by polymerization of one type of monomer or by polymerization of several types of monomer which are structurally different from each other. If the polymer is produced by polymerizing a type of monomer, it is called a homo-polymer. If structurally different monomer types are used in polymerization, the resulting polymer is called a copolymer.

The maximum molecular weight of the polymer depends on the degree of polymerization (number of polymerized monomers) and the batch size and is determined by the polymerization method. For the purposes of the present disclosure, it is preferred that the maximum molecular weight of the film-forming hydrophobic polymer (c) is not more than about 10⁷ g/mol, preferably not more than about 10⁶ g/mol and particularly preferably not more than about 10⁵ g/mol.

The composition (A) used in the process of the present disclosure may contain the condensation product of a C₁-C₆ organic alkoxy silane. The condensation products (A1) are oligomeric compounds that do not fall under the definition of a polymer. However, it cannot be completely ruled out that under exceptional storage conditions, such as particularly long storage times or remarkably elevated temperatures, a small proportion of the C₁-C₆ alkoxy silanes in composition (A) may condense not only to oligomeric but also to polymeric compounds.

Furthermore, the composition (B) used in the process of the present disclosure may contain a protein hydrolysate. Protein hydrolysates (B1) are also oligomeric compounds that do not fall under the definition of a polymer. In the case of protein hydrolysates (B1), however, it cannot be categorically ruled out that with the purchase of a corresponding purchasable protein hydrolysate (B1), a raw material is supplied which may also contain, as a by-product, portions of a high molecular weight protein in small quantities.

In other words, a first object of the present disclosure is a method for treating keratinous material, in particular human hair, wherein on the keratinous material are applied:

a first composition (A) comprising:

(A1) one or more organic C₁-C₆ alkoxy silanes and/or condensation products thereof, and

a second composition (B) comprising

(B1) at least one compound selected from the group of amino acids and protein hydrolysates,
where

the total content of all polymers other than condensation products (A1) contained in the composition (A)—based on the total weight of the composition (A)—is below about 0.3% by weight, and

the total content of all polymers other than protein hydrolysates (B1) contained in the composition (B)—based on the total weight of the composition (B)—is below about 0.3% by weight.

In other words, a first object of the present disclosure is a method for treating keratinous material, in particular human hair, wherein on the keratinous material are applied:

a first composition (A) comprising:

(A1) one or more organic C₁-C₆ alkoxy silanes and/or condensation products thereof, and

a second composition (B) comprising

(B1) at least one compound selected from the group of amino acids and protein hydrolysates,
where

the total content of all polymers other than the compounds of group (A1) present in the composition (A) is below about 0.3% by weight, based on the total weight of the composition (A), and

the total content of all polymers contained in the composition (B) which are different from the compounds of group (B1)—based on the total weight of the composition (B)—is below about 0.3% by weight.

Even if the polymer content in compositions (A) and (B) is limited to a maximum content of less than about 0.3 wt. %, good color results could be obtained in multiple dyeing. However, even higher color intensities were observed when the polymer content in composition (A) and in composition (B) was limited to even smaller amounts.

If the total content of all polymers contained in composition (A)—based on the total weight of composition (A)—was limited to a value below about 0.2 wt. %, preferably below about 0.1 wt. % and most preferably below about 0.01 wt. %, the results could be further improved.

In a further particularly preferred embodiment, a process according to the present disclosure is therefore exemplified in that the total content of all polymers presents in composition (A)—based on the total weight of composition (A)—is below about 0.2% by weight, preferably below about 0.1% by weight and very particularly preferably below about 0.01% by weight.

The same applies to the composition (B). If the total content of all polymers contained in composition (B)—based on the total weight of composition (B)—was limited to a value below about 0.2 wt. %, preferably below about 0.1 wt. % and most preferably below about 0.01 wt. %, the results could be further improved.

In another very particularly preferred embodiment, a process according to the present disclosure is therefore exemplified in that the total content of all polymers presents in the composition (B)—based on the total weight of the composition (B)—is below about 0.2% by weight, preferably below about 0.1% by weight and very particularly preferably below about 0.01% by weight.

Particularly preferably, compositions (A) and (B) are free of film-forming compounds. The film-forming polymers can be hydrophilic or hydrophobic.

In a further embodiment, it may be preferred to omit the use of a hydrophobic film-forming polymer in the preparation (A) and/or (B).

A hydrophobic polymer is a polymer that has a solubility in water at 25° C. (760 mmHg) of less than 1% by weight.

The water solubility of the film-forming, hydrophobic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1% by weight.

These include acrylic acid-type polymers, polyurethanes, polyesters, polyamides, polyureas, cellulose polymers, nitrocellulose polymers, silicone polymers, acrylamide-type polymers and polyisoprenes.

Film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

As further film-forming hydrophobic polymers may be mentioned the compounds selected from the group of synthetic polymers, polymers obtainable by radical polymerization or natural polymers.

Other film-forming hydrophobic polymers may be selected from the homopolymers or copolymers of olefins, such as cycloolefins, butadiene, isoprene or styrene, vinyl ethers, vinyl amides, the esters or amides of (meth)acrylic acid having at least one C₁-C₂₀ alkyl group, an aryl group or a C₂-C₁₀ hydroxyalkyl group.

Other film-forming hydrophobic polymers may be selected from the homo- or copolymers of isooctyl (meth)acrylate; isonononyl (meth)acrylate; 2-ethylhexyl (meth)acrylate; lauryl (meth)acrylate; isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate and/or mixtures thereof.

Other film-forming hydrophobic polymers may be selected from the homo- or copolymers of (meth)acrylamide; N-alkyl-(meth)acrylamides, in those with C2-C18 alkyl groups, such as N-ethyl-acrylamide, N-tert-butyl-acrylamide, le N-octyl-acrylamide; N-di(C1-C4)alkyl-(meth)acrylamide.

Examples of anionic polymers are the copolymers of acrylic acid, methacrylic acid or their C₁-C₆ alkyl esters, as sold under the INCI declaration Acrylates Copolymers. One commercial product, for example, is Aculyn® 33 from Rohm & Haas. Other polymers include copolymers of acrylic acid, methacrylic acid or their C₁-C₆ alkyl esters and the esters of an ethylenically unsaturated acid and an alkoxylated fatty alcohol.

Polymers on the market include Aculyn® 22 (Acrylate/Steareth-20 Methacrylate Copolymer), Aculyn® 28 (Acrylate/Beheneth-25 Methacrylate Copolymer), Structure 2001® (Acrylates/Steareth-20 Itaconate Copolymer), Structure 3001® (Acrylate/Ceteth-20 Itaconate Copolymer), Structure Plus® (acrylate/aminoacrylate C10-30 alkyl PEG-20 itaconate copolymer), Carbopol® 1342, 1382, Ultrez 20, Ultrez 21 (acrylate/C10-30 alkyl acrylate crosspolymer), Synthalen W 2000® (acrylate/palmeth-25 acrylate copolymer) or Soltex® OPT (acrylate/C12-22 alkyl methacrylate copolymer) distributed by Rohme and Haas.

Polymers based on vinyl monomers may include, for example, the homopolymers and copolymers of N-vinylpyrrolidone, vinylcaprolactam, vinyl-(C1-C6)alkyl-pyrrole, vinyl-oxazole, vinyl-thiazole, vinylpyrimidine, vinylimidazole.

Furthermore, the copolymers octylacrylamide/acrylates/butylaminoethyl methacrylate copolymer, as commercially sold for example under the trade names AMPHOMER® or LOVOCRYL® 47 by NATIONAL STARCH, or the copolymers of acrylates/octylacrylamides sold under the trade names DERMACRYL® LT and DERMACRYL® 79 by NATIONAL STARCH can be mentioned.

Polymers based on olefins include, for example, the homopolymers and copolymers of ethylene, propylene, butene, isoprene and butadiene.

Other film-forming hydrophobic polymers are the block copolymers comprising at least one block of styrene or the derivatives of styrene. These block copolymers can be copolymers that contain one or more other blocks in addition to a styrene block, such as styrene/ethylene, styrene/ethylene/butylene, styrene/butylene, styrene/isoprene, styrene/butadiene. Such polymers are commercially distributed by BASF® under the trade name “Luvitol HSB”.

All these polymers are used in the process according to the present disclosure in compositions (A) and (B) at most in the maximum amounts described above.

Other polymers are hydrophilic, film-forming polymers.

A hydrophilic polymer is a polymer that has a solubility in water at 25° C. (760 mmHg) of more than 1% by weight, preferably more than 2% by weight.

The water solubility of the film-forming, hydrophilic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, the solubility of the polymer is more than 1% by weight.

Nonionic, anionic and cationic polymers can be named as film-forming, hydrophilic polymers.

For example, film-forming, hydrophilic polymers from the group of polyvinylpyrrolidone (co)polymers, polyvinyl alcohol (co)polymers, vinyl acetate (co)polymers, the carboxyvinyl (co)polymers, the acrylic acid (co)polymers, the methacrylic acid (co)polymers, the natural gums, the polysaccharides and/or the acrylamide (co)polymers.

Polyvinylpyrrolidone (PVP) can also be mentioned as a film-forming, hydrophilic polymer.

Corresponding polyvinylpyrrolidones are available, for example, under the name Luviskol® K from BASF® SE, Luviskol® K 90 or Luviskol® K 85 from BASF® SE.

Another polyvinylpyrrolidone (PVP) that can be mentioned is the polymer PVP K30, which is marketed by Ashland® (ISP, POI Chemical). PVP K 30 is a polyvinylpyrrolidone which is highly soluble in cold water and has the CAS number 9003-39-8. The molecular weight of PVP K 30 is about 40000 g/mol.

Other polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115, which are available from BASF®.

Other film-forming, hydrophilic polymers that can be mentioned in this context are vinylpyrrolidone-vinyl ester copolymers, such as those sold under the trademark Luviskol® (BASF®). Luviskol® VA 64 and Luviskol® VA 73 are both vinylpyrrolidone/vinyl acetate copolymers.

The group of vinylpyrrolidone-containing copolymers also includes styrene/VP copolymer and/or a vinylpyrrolidone-vinyl acetate copolymer and/or a VP/DMAPA acrylates copolymer and/or a VP/vinyl caprolactam/DMAPA acrylates copolymer.

Vinylpyrrolidone-vinyl acetate copolymers are marketed under the name Luviskol® VA by BASF® SE. For example, a VP/Vinyl Caprolactam/DMAPA Acrylates copolymer is sold under the trade name Aquaflex® SF-40 by Ashland® Inc. For example, a VP/DMAPA acrylates copolymer is marketed as Styleze® CC-10 by Ashland®.

Other suitable copolymers of polyvinylpyrrolidone may also be those obtained by reacting N-vinylpyrrolidone with at least one further monomer from the group of V-vinylformamide, vinyl acetate, ethylene, propylene, acrylamide, vinylcaprolactam, vinylcaprolactone and/or vinyl alcohol.

Another copolymer of vinylpyrrolidone is the polymer known under the INCI designation maltodextrin/VP copolymer.

According to the present disclosure, a non-ionic polymer is understood to be a polymer which in a protic solvent—such as water—under standard conditions does not carry structural units with permanent cationic or anionic groups, which must be compensated by counterions while maintaining electron neutrality. Cationic groups include quaternized ammonium groups but not protonated amines. Anionic groups include carboxylic and sulphonic acid groups.

Appropriate nonionic, film-forming, hydrophilic polymers are selected from the group of.

Polyvinylpyrrolidone,

Copolymers of N-vinylpyrrolidone and vinyl esters of carboxylic acids having 2 to about 18 carbon atoms of N-vinylpyrrolidone and vinyl acetate,

Copolymers of N-vinylpyrrolidone and N-vinylimidazole and methacrylamide,

Copolymers of N-vinylpyrrolidone and N-vinylimidazole and acrylamide,

Copolymers of N-vinylpyrrolidone with N,N-di(C1 to C4)-alkylamino-(C2 to C4)-alkylacrylamide.

If copolymers of N-vinylpyrrolidone and vinyl acetate are used, the molar ratio of the structural units contained from the monomer N-vinylpyrrolidone to the structural units of the polymer contained from the monomer vinyl acetate can be in the range from about 20 to about 80 to about 80 to about 20, or from about 30 to about 70 to about 60 to about 40. Suitable copolymers of vinyl pyrrolidone and vinyl acetate are available, for example, under the trademarks Luviskol® VA 37, Luviskol® VA 55, Luviskol® VA 64 and Luviskol® VA 73 from BASF® SE.

A further polymer is selected from polymers with the INCI designation VP/Methacrylamide/Vinyl Imidazole Copolymer, which are available, for example, under the trade name Luviset® Clear from BASF® SE.

Another non-ionic, film-forming, hydrophilic polymer is a copolymer of N-vinylpyrrolidone and N,N-dimethylaminiopropylmethacrylamide, which is sold, for example, by ISP under the INCI designation VP/DMAPA Acrylates Copolymer, e.g., under the trade name Styleze® CC 10.

A cationic ePolymer is the copolymer of N-vinylpyrrolidone, N-vinylcaprolactam, N-(3-dimethylaminopropyl)methacrylamide, and 3-(methacryloylamino)propyl-lauryl-dimethylammonium chloride (INCI name: Polyquaternium-69), which is marketed, for example, under the trade name AquaStyle® 300 (28-32 wt. % active substance in ethanol-water mixture, molecular weight 350000) by ISP.

Other film-forming hydrophilic polymers include.
Vinylpyrrolidone-vinylimidazolium methochloride copolymers, as offered under the designations Luviquat® FC 370, FC 550 and the INCI designation Polyquaternium-16 as well as FC 905 and HM 552,
Vinylpyrrolidone-vinylcaprolactam-acrylate terpolymers, as they are commercially available with acrylic acid esters and acrylic acid amides as a third monomer component, for example under the name Aquaflex® SF 40.

Polyquaternium-11 is the reaction product of diethyl sulphate with a copolymer of vinyl pyrrolidone and dimethylaminoethyl methacrylate. Suitable commercial products are available under the names Dehyquart® CC 11 and Luviquat® PQ 11 PN from BASF® SE or Gafquat® 440, Gafquat® 734, Gafquat® 755 or Gafquat® 755N from Ashland® Inc.

Polyquaternium-46 is the reaction product of vinylcaprolactam and vinylpyrrolidone with methylvinylimidazolium methosulfate and is available for example under the name Luviquat® Hold from BASF® SE.

As anionic film-forming, hydrophilic polymers, for example, acrylic acid polymers can be mentioned, which can be present in uncrosslinked or crosslinked form. Such products are sold commercially under the trade names Carbopol® 980, 981, 954, 2984 and 5984 by Lubrizol® or under the names Synthalen® M and Synthalen® K by 3V Sigma (The Sun Chemicals®, Inter Harz).

Examples of suitable film-forming, hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Film-forming, hydrophilic polymers from the group of acrylamides are, for example, polymers which are prepared starting from monomers of (methyl)acrylamido-C1-C4-alkyl-sulfonic acid or salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Cross-linked and fully or partially neutralized polymers of the poly-2-acrylamido-2-methylpropane sulfonic acid type are available under the INCI designation “Ammonium Polyacrylamido-2-methyl-propanesulphonates” or “Ammonium Polyacryldimethyltauramides”.

Another polymer of this type is the crosslinked poly-2-acrylamido-2methyl-propanesulfonic acid polymer sold by Clamant under the trade name Hostacerin® AMPS, which is partially neutralized with ammonia.

All these polymers are used in the process according to the present disclosure in compositions (A) and (B) at most in the maximum amounts described above.

### Process for Dyeing Keratin Material

In the course of the work leading to the present disclosure, it was observed that the use of compositions (A) and (B) in a dyeing process results in dyeings with particularly high color intensity and good wash fastness. Especially with repeated application satisfactory results could be obtained.

If the process according to the present disclosure is a process for coloring keratin material, at least one process step comprises the application of at least one coloring compound, in particular at least one pigment. In this case, it is possible to incorporate the pigment into the composition (A). It is also possible to add at least one pigment to composition (B). Furthermore, it is also according to the present disclosure if the colorant compound, in particular the pigment, is incorporated into a third composition (C), which can be applied to the keratin material, for example, before or after composition (A).

It has been found to be particularly preferred if the first composition (A) additionally contains at least one colorant compound selected from the group of pigments and direct dyes.

In an explicitly quite particularly preferred embodiment, a process according to the present disclosure is exemplified in that the first composition (A) comprises at least one colorant compound from the group of pigments and/or direct dyes.

Furthermore, it has also been found to be particularly preferred if the second composition (B) additionally contains at least one colorant compound selected from the group of pigments and direct dyes.

In an explicitly quite particularly preferred embodiment, a process according to the present disclosure is exemplified in that the second composition (B) comprises at least one colorant compound from the group of pigments and/or direct dyes.

The colorant compound(s) can preferably be selected from pigments, direct dyes, where direct dyes can also be photochromic dyes and thermochromic dyes.

Very preferably, composition (A) and/or composition (B) contains at least one pigment.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the finely dispersed pigment, the mixture is filtered. If a proportion of undissolved pigments remains on the filter paper, the solubility of the pigment is below 0.5 g/L.

Suitable color pigments can be of inorganic and/or organic origin.

In a preferred embodiment, a composition according to the present disclosure is exemplified in that it comprises at least one colorant compound selected from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred colorants from the group of pigments according to the present disclosure. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, muscovite or phlogopite, is coated with a metal oxide.

In a very particularly preferred embodiment, a process according to the present disclosure is exemplified in that the composition (A) and/or the composition (B) comprises at least one colorant compound from the group of inorganic pigments selected from the group of colored metal oxides, metal hydroxides, metal oxide hydrates, silicates, metal sulfides, complex metal cyanides, metal sulfates, bronze pigments and/or colored mica- or mica-based pigments coated with at least one metal oxide and/or a metal oxychloride.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, the composition (A) according to the present disclosure and/or the composition (B) is exemplified in that it comprises at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a composition (A) and/or composition (B) according to the present disclosure is exemplified in that it comprises at least one colorant compound selected from mica- or mica-based pigments which are reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck®, Ariabel® and Unipure® from Sensient®, Prestige® from Eckart® Cosmetic Colors and Sunshine® from Sunstar®.

Particularly preferred color pigments with the trade name Colorona® are, for example:
Colorona® Copper, Merck®, MICA, CI 77491 (IRON OXIDES)
Colorona® Passion Orange, Merck®, Mica, CI 77491 (Iron Oxides), Alumina
Colorona® Patina Silver, Merck®, MICA, CI 77499 (IRON OXIDES), CI 77891 (TITANIUM DIOXIDE)
Colorona® RY, Merck®, CI 77891 (TITANIUM DIOXIDE), MICA, CI 75470 (CARMINE)
Colorona® Oriental Beige, Merck®, MICA, CI 77891 (TITANIUM DIOXIDE), CI 77491 (IRON OXIDES)
Colorona® Dark Blue, Merck®, MICA, TITANIUM DIOXIDE, FERRIC FERROCYANIDE
Colorona® Chameleon, Merck®, CI 77491 (IRON OXIDES), MICA
Colorona® Aborigine Amber, Merck®, MICA, CI 77499 (IRON OXIDES), CI 77891 (TITANIUM DIOXIDE)
Colorona® Blackstar Blue, Merck®, CI 77499 (IRON OXIDES), MICA
Colorona® Patagonian Purple, Merck®, MICA, CI 77491 (IRON OXIDES), CI 77891 (TITANIUM DIOXIDE), CI 77510 (FERRIC FERROCYANIDE)
Colorona® Red Brown, Merck®, MICA, CI 77491 (IRON OXIDES), CI 77891 (TITANIUM DIOXIDE)
Colorona® Russet, Merck®, CI 77491 (TITANIUM DIOXIDE), MICA, CI 77891 (IRON OXIDES)
Colorona® Imperial Red, Merck®, MICA, TITANIUM DIOXIDE (CI 77891), D&C RED NO. 30 (CI 73360)
Colorona® Majestic Green, Merck®, CI 77891 (TITANIUM DIOXIDE), MICA, CI 77288 (CHROMIUM OXIDE GREENS)
Colorona® Light Blue, Merck®, MICA, TITANIUM DIOXIDE (CI 77891), FERRIC FERROCYANIDE (CI 77510)
Colorona® Red Gold, Merck®, MICA, CI 77891 (TITANIUM DIOXIDE), CI 77491 (IRON OXIDES)
Colorona® Gold Plus MP 25, Merck®, MICA, TITANIUM DIOXIDE (CI 77891), IRON OXIDES (CI 77491)
Colorona® Carmine Red, Merck®, MICA, TITANIUM DIOXIDE, CARMINE
Colorona® Blackstar Green, Merck®, MICA, CI 77499 (IRON OXIDES)
Colorona® Bordeaux, Merck®, MICA, CI 77491 (IRON OXIDES)
Colorona® Bronze, Merck®, MICA, CI 77491 (IRON OXIDES)
Colorona® Bronze Fine, Merck®, MICA, CI 77491 (IRON OXIDES)
Colorona® Fine Gold MP 20, Merck®, MICA, CI 77891 (TITANIUM DIOXIDE), CI 77491 (IRON OXIDES)
Colorona® Sienna Fine, Merck®, CI 77491 (IRON OXIDES), MICA
Colorona® Sienna, Merck®, MICA, CI 77491 (IRON OXIDES)
Colorona Precious Gold, Merck®, Mica, CI 77891 (Titanium dioxide), Silica, CI 77491(Iron oxides), Tin oxide
Colorona® Sun Gold Sparkle MP 29, Merck®, MICA, TITANIUM DIOXIDE, IRON OXIDES, MICA, CI 77891, CI 77491 (EU)
Colorona® Mica Black, Merck®, CI 77499 (Iron oxides), Mica, CI 77891 (Titanium dioxide)
Colorona® Bright Gold, Merck®, Mica, CI 77891 (Titanium dioxide), CI 77491(Iron oxides)
Colorona® Blackstar Gold, Merck®, MICA, CI 77499 (IRON OXIDES)

Other particularly preferred color pigments with the trade name Xirona® are for example:
Xirona® Golden Sky, Merck®, Silica, CI 77891 (Titanium Dioxide), Tin Oxide
Xirona® Caribbean Blue, Merck®, Mica, CI 77891 (Titanium Dioxide), Silica, Tin Oxide
Xirona® Kiwi Rose, Merck®, Silica, CI 77891 (Titanium Dioxide), Tin Oxide Xirona® Magic Mauve, Merck®, Silica, CI 77891 (Titanium Dioxide), Tin Oxide.

In addition, particularly preferred color pigments with the trade name Unipure® are for example:
Unipure® Red LC 381 EM, Sensient® CI 77491 (Iron Oxides), Silica
Unipure® Black LC 989 EM, Sensient®, CI 77499 (Iron Oxides), Silica
Unipure® Yellow LC 182 EM, Sensient®, CI 77492 (Iron Oxides), Silica

In the context of a further embodiment, composition (A) and/or composition (B) may also comprise one or more color-imparting compounds selected from the group of organic pigments.

The organic pigments according to the present disclosure are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindigo, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, a process according to the present disclosure is exemplified in that the composition (A) and/or the composition (B) comprises at least one colorant compound from the group of organic pigments selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. In the sense of the present disclosure, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent resistance to light and temperature, the use of the pigments in the present disclosure is particularly preferred. It is also preferred if the pigments used have a certain particle size. This particle size leads on the one hand to an even distribution of the pigments in the formed polymer film and on the other hand avoids a rough hair or skin feeling after application of the cosmetic product. According to the present disclosure, it is therefore advantageous if the at least one pigment has an average particle size D₅₀ of about 1.0 to about 50 μm, preferably about 5.0 to about 45 μm, preferably about 10 to about 40 μm, or from about 14 to about 30 μm. The mean particle size D₅₀, for example, can be determined using dynamic light scattering (DLS).

Pigments with a specific shaping may also have been used to color the keratin material. For example, a pigment based on a lamellar and/or a lenticular substrate platelet can be used. Furthermore, coloring based on a substrate platelet comprising a vacuum metallized pigment is also possible.

The substrate platelets of this type have an average thickness of at most about 50 nm, preferably less than about 30 nm, particularly preferably at most about 25 nm, for example at most about 20 nm. The average thickness of the substrate platelets is at least about 1 nm, preferably at least about 2.5 nm, particularly preferably at least about 5 nm, for example at least about 10 nm. Preferred ranges for substrate wafer thickness are about 2.5 to about 50 nm, about 5 to about 50 nm, about 10 to about 50 nm; about 2.5 to about 30 nm, about 5 to about 30 nm, about 10 to about 30 nm; about 2.5 to about 25 nm, about 5 to about 25 nm, about 10 to about 25 nm, about 2.5 to about 20 nm, about 5 to about 20 nm, and about 10 to about 20 nm. Preferably, each substrate plate has a thickness that is as uniform as possible.

Due to the low thickness of the substrate platelets, the pigment exhibits particularly high hiding power.

The substrate plates have a monolithic structure. Monolithic in this context means including a single closed unit without fractures, stratifications or inclusions, although structural changes may occur within the substrate platelets. The substrate platelets are preferably homogeneously structured, i.e., there is no concentration gradient within the platelets. In particular, the substrate platelets do not have a layered structure and do not have any particles or particles distributed in them.

The size of the substrate platelet can be adjusted to the respective application purpose, especially the desired effect on the keratinic material. Typically, the substrate platelets have an average largest diameter of about 2 to about 200 μm, especially about 5 to about 100 μm.

In a preferred design, the aspect ratio, expressed by the ratio of the average size to the average thickness, is at least about 80, preferably at least about 200, more preferably at least about 500, more preferably more than about 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for 3 minutes.

The substrate platelets can be composed of any material that can be formed into platelet shape.

They can be of natural origin, but also synthetically produced. Materials from which the substrate platelets can be constructed include metals and metal alloys, metal oxides, preferably aluminum oxide, inorganic compounds and minerals such as mica and (semi-)precious stones, and plastics. Preferably, the substrate platelets are constructed of metal (alloy).

Any metal suitable for metallic luster pigments can be used. Such metals include iron and steel, as well as all air and water resistant (semi)metals such as platinum, zinc, chromium, molybdenum and silicon, and their alloys such as aluminum bronzes and brass. Preferred metals are aluminum, copper, silver and gold. Preferred substrate platelets include aluminum platelets and brass platelets, with aluminum substrate platelets being particularly preferred.

Lamellar substrate platelets are exemplified by an irregularly structured edge and are also referred to as “cornflakes” due to their appearance.

Due to their irregular structure, pigments based on lamellar substrate platelets generate a high proportion of scattered light. In addition, pigments based on lamellar substrate platelets do not completely cover the existing color of a keratinous material, and effects analogous to natural graying can be achieved, for example.

Lenticular (=lens-shaped) substrate platelets have a regular round edge and are also called “silver dollars” due to their appearance. Due to their regular structure, the proportion of reflected light predominates in pigments based on lenticular substrate platelets.

Vacuum metallized pigments (VMP) can be obtained, for example, by releasing metals, metal alloys or metal oxides from suitably coated films. They are exemplified by a particularly low thickness of the substrate platelets in the range of about 5 to about 50 nm and a particularly smooth surface with increased reflectivity. Substrate platelets comprising a vacuum metallized pigment are also referred to as VMP substrate platelets in the context of this application. VMP substrate platelets of aluminum can be obtained, for example, by releasing aluminum from metallized films.

The metal or metal alloy substrate plates can be passivated, for example by anodizing (oxide layer) or chromating.

Uncoated lamellar, lenticular and/or VPM substrate plates, especially those made of metal or metal alloy, reflect the incident light to a high degree and create a light-dark flop but no color impression.

A color impression can be created by optical interference effects, for example. Such pigments can be based on at least single-coated substrate platelets. These show interference effects by superimposing differently refracted and reflected light beams.

Accordingly, preferred pigments are pigments based on a coated lamellar substrate platelet. The substrate wafer preferably has at least one coating B of a highly refractive metal oxide having a coating thickness of at least about 50 nm. There is preferably another coating A between the coating B and the surface of the substrate wafer. If necessary, there is a further coating C on the layer B, which is different from the layer B underneath.

Suitable materials for coatings A, B and C are all substances that can be applied to the substrate platelets in a film-like and permanent manner and, in the case of coatings A and B, have the required optical properties. Coating part of the surface of the substrate platelets is sufficient to obtain a pigment with a glossy effect. For example, only the top and/or bottom of the substrate platelets may be coated, with the side surface(s) omitted. Preferably, the entire surface of the optionally passivated substrate platelets, including the side surfaces, is covered by coating B. The substrate platelets are thus completely enveloped by coating B. This improves the optical properties of the pigment and increases its mechanical and chemical resistance. The above also applies to layer A and preferably also to layer C, if present.

Although multiple coatings A, B and/or C may be present in each case, the coated substrate wafers preferably have only one coating A, B and, if present, C in each case.

The coating B is composed of at least one highly refractive metal oxide. Highly refractive materials have a refractive index of at least about 1.9, preferably at least about 2.0, and more preferably at least about 2.4. Preferably, the coating B comprises at least about 95 wt. %, more preferably at least about 99 wt. %, of high refractive index metal oxide(s).

The coating B has a thickness of at least about 50 nm. Preferably, the thickness of coating B is no more than about 400 nm, more preferably no more than about 300 nm.

Highly refractive metal oxides suitable for coating B are preferably selectively light-absorbing (i.e., colored) metal oxides, such as iron(III) oxide (α- and γ-Fe203, red), cobalt(II) oxide (blue), chromium(III) oxide (green),titanium(III) oxide (blue, usually present in admixture with titanium oxynitrides and titanium nitrides), and vanadium(V) oxide (orange), and mixtures thereof. Colorless high-index oxides such as titanium dioxide and/or zirconium oxide are also suitable.

Coating B may contain a selectively absorbing dye, preferably about 0.001 to about 5% by weight, particularly preferably about 0.01 to about 1% by weight, in each case based on the total amount of coating B. Suitable dyes are organic and inorganic dyes which can be stably incorporated into a metal oxide coating.

The coating A preferably has at least one low refractive index metal oxide and/or metal oxide hydrate. Preferably, coating A comprises at least about 95 wt. %, more preferably at least about 99 wt. %, of low refractive index metal oxide (hydrate). Low refractive index materials have a refractive index of about 1.8 or less, preferably about 1.6 or less.

Low refractive index metal oxides suitable for coating A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. The coating A preferably has a thickness of about 1 to about 100 nm, particularly preferably about 5 to about 50 nm, especially preferably about 5 to about 20 nm.

Preferably, the distance between the surface of the substrate platelets and the inner surface of coating B is at most about 100 nm, particularly preferably at most about 50 nm, especially preferably at most about 20 nm. By ensuring that the thickness of coating A, and thus the distance between the surface of the substrate platelets and coating B, is within the range specified above, it is possible to ensure that the pigments have a high hiding power.

If the pigment based on a lamellar substrate platelet has only one layer A, it is preferred that the pigment has a lamellar substrate platelet of aluminum and a layer A of silica. If the pigment based on a lamellar substrate platelet has a layer A and a layer B, it is preferred that the pigment has a lamellar substrate platelet of aluminum, a layer A of silica and a layer B of iron oxide.

According to a preferred embodiment, the pigments have a further coating C of a metal oxide (hydrate), which is different from the underlying coating B. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

The coating C preferably has a thickness of about 10 to about 500 nm, more preferably about 50 to about 300 nm. By providing coating C, for example based on TiO₂, better interference can be achieved while maintaining high hiding power.

Layers A and C serve as corrosion protection as well as chemical and physical stabilization. Particularly preferred layers A and C are silica or alumina applied by the sol-gel process. This process comprises dispersing the uncoated lamellar substrate platelets or the lamellar substrate platelets already coated with layer A and/or layer B in a solution of a metal alkoxide such as tetraethyl orthosilicate or aluminum triisopropanolate (usually in a solution of organic solvent or a mixture of organic solvent and water with at least about 50% by weight of organic solvent such as a C1 to C4 alcohol) and adding a weak base or acid to hydrolyze the metal alkoxide, thereby forming a film of the metal oxide on the surface of the (coated) substrate platelets.

Layer B can be produced, for example, by hydrolytic decomposition of one or more organic metal compounds and/or by precipitation of one or more dissolved metal salts, as well as any subsequent post-treatment (for example, transfer of a formed hydroxide-containing layer to the oxide layers by annealing).

Although each of the coatings A, B and/or C may be composed of a mixture of two or more metal oxide(hydrate)s, each of the coatings is preferably composed of one metal oxide(hydrate).

The pigments based on coated lamellar or lenticular substrate platelets, or the pigments based on coated VMP substrate platelets preferably have a thickness of about 70 to about 500 nm, particularly preferably about 100 to about 400 nm, especially preferably about 150 to about 320 nm, for example about 180 to about 290 nm. Due to the low thickness of the substrate platelets, the pigment exhibits particularly high hiding power. The low thickness of the coated substrate platelets is achieved by keeping the thickness of the uncoated substrate platelets low, but also by adjusting the thicknesses of the coatings A and, if present, C to as small a value as possible. The thickness of coating B determines the color impression of the pigment.

The adhesion and abrasion resistance of pigments based on coated substrate platelets in keratinic material can be significantly increased by additionally modifying the outermost layer, layer A, B or C depending on the structure, with organic compounds such as silanes, phosphoric acid esters, titanates, borates or carboxylic acids. In this case, the organic compounds are bonded to the surface of the outermost, preferably metal oxide-containing, layer A, B, or C. The outermost layer denotes the layer that is spatially farthest from the lamellar substrate platelet. The organic compounds are preferably functional silane compounds that can bind to the metal oxide-containing layer A, B, or C. These can be either mono- or bifunctional compounds. Examples of bifunctional organic compounds include methacryloxypropenyltrimethoxysilane, 3-methacryloxypropyltrimethoxysilane, 3-acryloxypropyltrimethoxysilane, 2-acryloxyethyltrimethoxysilane, 3 -methacryloxy-propyltriethoxysilane, 3-acryloxypropyltrimethoxysilane, 2-methacryloxyethyl-triethoxysilane, 2-acryloxyethyltriethoxysilane, 3-methacryloxypropyltris(methoxyethoxy)silane, 3-methacryloxypropyltris(butoxyethoxy)silane, 3-methacryloxypropyltris(propoxy)silane, 3 -methacryloxypropyltris(butoxy)silane, 3 -acryloxy-propyltris(methoxyethoxy)silane, 3-acryloxypropyltris(butoxyethoxy)silane, 3-acryl-oxypropyltris(butoxy)silane, vinyltrimethoxysilane, vinyltriethoxysilane, vinylethyl dichlorosilane, vinylmethyldiacetoxysilane, vinylmethyldichlorosilane, vinylmethyldiethoxysilane, vinyltriacetoxysilane, vinyltrichlorosilane, phenylvinyldiethoxysilane, or phenylallyldichlorosilane. Furthermore, a modification with a monofunctional silane, an alkyl silane or aryl silane, can be carried out. This has only one functional group, which can covalently bond to the surface pigment based on coated lamellar substrate platelets (i.e., to the outermost metal oxide-containing layer) or, if not completely covered, to the metal surface. The hydrocarbon residue of the silane points away from the pigment. Depending on the type and nature of the hydrocarbon residue of the silane, a varying degree of hydrophobicity of the pigment is achieved. Examples of such silanes include hexadecyltrimethoxysilane, propyltrimethoxysilane, etc. Particularly preferred are pigments based on silica-coated aluminum substrate platelets surface-modified with a monofunctional silane. Octyltrimethoxysilane, octyltriethoxysilane, hecadecyltrimethoxysilane and hecadecyltriethoxysilane are particularly preferred. Due to the changed surface properties/hydrophobization, an improvement can be achieved in terms of adhesion, abrasion resistance and alignment in the application.

Suitable pigments based on a lamellar substrate platelet include, for example, the pigments of the VISIONAIRE series from Eckart®.

Pigments based on a lenticular substrate platelet are available, for example, under the name Alegrace® Gorgeous from the company Schlenk® Metallic Pigments GmbH.

Pigments based on a substrate platelet comprising a vacuum metallized pigment are available, for example, under the name Alegrace® Marvelous or Alegrace® Aurous from the company Schlenk® Metallic Pigments GmbH.

In a further embodiment, a process according to the present disclosure is exemplified in that the composition (A) contains—based on the total weight of the composition (A)—one or more pigments in a total amount of from about 0.001 to about 20% by weight, from about 0.05 to about 5% by weight.

In a further embodiment, a process according to the present disclosure is exemplified in that the composition (B) contains—based on the total weight of the composition (B)—one or more pigments in a total amount of from about 0.001 to about 20% by weight, from about 0.05 to about 5% by weight.

As colorant compounds, the compositions according to the present disclosure may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments. Preferably, the direct dyes within the meaning of the present disclosure have a solubility in water (760 mmHg) at 25° C. of more than 1.0 g/L. In particular, the direct dyes within the meaning of the present disclosure have a solubility in water (760 mmHg) at 25° C. of more than 1.5 g/L.

Direct dyes can be divided into anionic, cationic and nonionic direct dyes.

In a further preferred embodiment, an agent according to the present disclosure is exemplified in that it contains at least one anionic, cationic and/or nonionic direct dye as the coloring compound.

In a further preferred embodiment, a process according to the present disclosure is exemplified in that the composition (B) and/or the composition (C) comprises at least one colorant compound selected from the group of anionic, nonionic, and/or cationic direct dyes.

Suitable cationic direct dyes include Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76.

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3 -nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—COOO⁻, —SO₃⁻ present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L. The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential feature of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group can be selected as particularly well suited acid dyes: Acid Yellow 1 (D&C Yellow 7, Citronin A, Ext. D&C Yellow No. 7, Japan Yellow 403,CI 10316, COLIPA n° B001), Acid Yellow 3 (COLIPA n°: C 54, D&C Yellow N° 10, Quinoline Yellow, E104, Food Yellow 13), Acid Yellow 9 (CI 13015), Acid Yellow 17 (CI 18965), Acid Yellow 23 (COLIPA n° C 29, Covacap Jaune W 1100 (LCW), Sicovit Tartrazine 85 E 102 (BASF®), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1;CI 20170;KATSU201;nosodiumsalt; Brown No.201; RESORCIN BROWN; ACID ORANGE 24; Japan Brown 201; D & C Brown No.1), Acid Red 14 (C.I.14720), Acid Red 18 (E124, Red 18; CI 16255), Acid Red 27 (E 123, CI 16185, C-Rot 46, Real red D, FD&C Red Nr.2, Food Red 9, Naphthol red S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI 27290), Acid Red 87 (Eosin, CI 45380), Acid Red 92 (COLIPA n° C53, CI 45410), Acid Red 95 (CI 45425, Erythtosine,Simacid Erythrosine Y), Acid Red 184 (CI 15685), Acid Red 195, Acid Violet 43 (Jarocol Violet 43, Ext. D&C Violet n° 2, C.I. 60730, COLIPA n° C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n° 401, Naphthalene Black 10B, Amido Black 10B, CI 20 470, COLIPA n° B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. A stir-fish is added. Then add 100 ml of water. This mixture is heated to 25° C. on a magnetic stirrer while stirring. It is stirred for 60 minutes. The aqueous mixture is then visually assessed. If there are still undissolved radicals, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.
Acid Yellow 1 is called 8-hydroxy-5,7-dinitro-2-naphthalenesulfonic acid disodium salt and has a solubility in water of at least 40 g/L (25° C.).
Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has a remarkably high water solubility of more than 20% by weight.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl}{4-[(N-ethyl(3-sulfonatobenzyl)imino]-2,5-cycohexadien-1-ylidene}methyl)-benzenesulfonate and has a solubility in water of more than 20% by weight (25° C.).

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to reversibly or irreversibly change its color depending on irradiation with light, especially UV light. This can be done by changing both the intensity and/or the wavelength maximum.

### Application of the Compositions (A) and (B)

The process according to the present disclosure comprises the application of both compositions (A) and (B) to the keratinous material. The two compositions (A) and (B) are two different compositions.

As described previously, it is particularly preferred if the composition (A) is first applied to the keratin material, and subsequently the composition (B) is applied to the keratin material in the form of an aftertreatment agent.

In the context of a further embodiment, particularly preferred is a method according to the present disclosure comprising the following steps:

(1) Application of the first composition (A) to the keratin material,

(2) Allowing the composition (A) to act on the keratin material for a period of about 1 to about 10 minutes, preferably about 1 to about 5 minutes,

(3) Rinsing the composition (A) out of the keratin material,

(4) Application of composition (B) to the keratin material,

(5) Allowing the composition (B) to act on the keratin material for a period of about 1 to about 10 minutes, preferably about 1 to about 5 minutes,

(6) Rinsing the composition (B) out of the keratin material.

The rinsing of the keratinous material with water in steps (3) and (6) of the process is understood according to the present disclosure to mean that only water is used for the rinsing process, without the use of other compositions different from compositions (a) and (b).

In a step (1), the composition (A) is first applied to the keratin materials, especially human hair.

After application, the composition (A) is allowed to act on the keratin materials. In this context, application times from about 10 seconds to about 10 minutes, preferably from about 20 seconds to about 5 minutes and especially preferably from about 30 seconds to about 2 minutes on the hair have proven to be particularly beneficial.

In a preferred embodiment of the process according to the present disclosure, the composition (A) can now be rinsed from the keratin materials before the composition (B) is applied to the hair in the subsequent step.

In step (4), the composition (B) is now applied to the keratin materials. After application, the composition (B) is now left to act on the hair.

The process according to the present disclosure allows the production of dyeings with particularly good intensity and wash fastness even with short exposure times of the compositions (A) and (B). Application times from about 10 seconds to about 10 minutes, preferably from about 20 seconds to about 5 minutes and most preferably from about 30 seconds to about 3 minutes on the hair have proven to be particularly beneficial.

In step (6), the composition (B) is now rinsed out of the keratin material with water.

In the context of a further embodiment, a method according to the present disclosure comprising the following steps in the order indicated is quite particularly preferred:

(1) Application of the first composition (A) to the keratin material,

(2) Allowing the composition (A) to act on the keratin material for a period of about 1 to about 10 minutes, preferably about 1 to about 5 minutes,

(3) Rinsing the composition (A) out of the keratin material,

(4) Application of composition (B) to the keratin material,

(5) Allowing the composition (B) to act on the keratin material for a period of about 1 to about 10 minutes, preferably about 1 to about 5 minutes,

(6) Rinsing the composition (B) out of the keratin material.

### Multi-Component Packaging Unit (Kit-of-Parts)

To increase user convenience, all preparations required for the application process, for the dyeing process, are provided to the user in the form of a multi-component packaging unit (kit-of-parts).

A second object of the present disclosure is a multi-component packaging unit (kit-of-parts) for treating keratinous material, comprising separately prepared
a first container comprising a first composition (A) and
a second container comprising a second composition (B), wherein
wherein the compositions (A) and (B) have already been disclosed in detail in the description of the first subject matter of the present disclosure.

Furthermore, the multi-component packaging unit according to the present disclosure may also comprise a third packaging unit containing a cosmetic preparation (C). The preparation (C) contains, as described above, very particularly preferably at least one coloring compound.

In a very particularly preferred embodiment, the multi-component packaging unit (kit-of-parts) according to the present disclosure comprises, separately assembled from one another

a third container comprising a third composition (C), the third composition (C) comprising at least one colorant compound selected from the group of pigments and/or direct dyes.

The colorant compounds from the group of pigments and direct dyes have already been disclosed in detail in the description of the first subject matter of the present disclosure.

With respect to the other preferred embodiments of the multi-component packaging unit according to the present disclosure, the same applies mutatis mutandis to the procedure according to the present disclosure.

1. A method of treating keratinous material, comprising:
applying a first composition (A) and a second composition (B) to the keratinous material: wherein;
the first composition (A) comprisingcomprises:
one or more organic C₁-C₆ alkoxy silanes (A1) and/or condensation products thereof, and
the second composition (B) comprises;
at least one compound (B1) selected from the group of amino acids, protein hydrolysates, or combinations thereof,
wherein
the total content of all polymers in the first composition (A)—based on a total weight of the first composition (A)—is below about 0.3% by weight, and
the total content of all polymers in the second composition (B) based on a total weight of the second composition (B)—is below about 0.3% by weight.

2. The method according to claim 1, wherein the first composition (A) comprises the one or more organic C₁-C₆ alkoxy silanes (A1) of the formula (S-I) and/or (S-II),
R₁R₂N-L-Si(OR₃)ₐ(R₄)_{b} (S-I)
where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a, stands for an integer from 1 to 3, and
b stands for the integer 3-a, and
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),
where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (S-III),
(A″″)-Si(R₆″)_{d}′(OR₅″)_{c}′ (S-III),
where
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0,
and/or their condensation products.)

3. The method according to claim 2, wherein the first composition (A) comprises the at least one C₁-C₆ organic alkoxysilane (A1) of formula (S-I) selected from the group of
(3 -Aminopropyl)triethoxysilane
(3 -Aminopropyl)trimethoxysilane
(2-Aminoethyl)triethoxysilane
(2-Aminoethyl)trimethoxysilane
(3 -Dimethylaminopropyl)triethoxysilane
(3 -Dimethylaminopropyl)trimethoxysilane
(2-Dimethylaminoethyl)triethoxysilane,
(2-Dimethylaminoethyl)trimethoxysilane
and/or their condensation products.)

4. The method according to claim 1, wherein the first composition (A) comprises the one or more organic C₁-C₆ alkoxy silanes (A1) of formula (S-IV),
R₉Si(OR₁₀)ₖ(R₁₁)ₘ (S-IV),
where
R₉ represents a C₁-C₁₂ alkyl group,
R₁₀ represents a C₁-C₆ alkyl group,
R₁₁ represents a C₁-C₆ alkyl group
k is an integer from 1 to 3, and
m stands for the integer 3-k,
and/or their condensation products.

5. The method according to claim 4, wherein the first composition (A) comprises the at least one C₁-C₆ organic alkoxysilane (A1) of formula (S-IV) selected from the group
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
and/or their condensation products.)

6. The method according to claim 1, wherein the first composition (A) comprises at least one cosmetic ingredient selected from the group of hexamethyldisiloxane, octamethyltrisiloxane, decamethyltetrasiloxane, hexamethylcyclotrisiloxane, octamethylcyclotetrasiloxane, decamethylcyclopentasiloxane, and combinations thereof.

7. The method according to claim 1, wherein the second composition (B) comprises the at least one amino acid of compound (B1) selected from the group of arginine, lysine, histidine, asparagine, glutamine, cysteine, methionine, tryptophan, serine, alanine, aspartic acid, glutamic acid, glycine, isoleucine, leucine, phenylalanine, proline, threonine, tyrosine, valine, and combinations thereof.

8. The method according to claim 1, wherein the second composition (B) comprises—based on the total weight of the second composition (B)—the one or more amino acids of compound (B1) in a total amount of about 0.1 to about 20.0% by weight.

9. The method according to claim 1, wherein the second composition (B) comprises the at least one protein hydrolysate of compound (B1) selected from the group consisting of protein hydrolysates of elastin, collagen, keratin, silk, milk protein, soy, almond, pea, moringa, potato and wheat protein hydrolysates, and combinations thereof.

10. The method according to claim 1, wherein the second composition (B) comprises—based on the total weight of the second composition (B)—the one or more protein hydrolysate of compound (B1) in a total amount of about 0.1 to about 20.0% by weight.

11. The method according to claim 1, wherein the second composition (B) has a pH of from about 7.0 to about 12.0.

12. The method according to claim 1, wherein the total content of all polymers presents in the first composition (A)—based on the total weight of the first composition (A)—is below about 0.2% by weight.

13. The method according to claim 1, wherein the total content of all polymers presents in the second composition (B)—based on the total weight of the second composition (B)—is below about 0.2% by weight.

14. The method according to claim 1, wherein the first composition (A) comprises at least one colorant compound selected from the group of pigments and/or direct dyes.

15. The method according to claim 1, wherein the second composition (B) comprises at least one colorant compound selected from the group of pigments and/or direct dyes.

16. The method of claim 1, comprising the following steps:
applying the first composition (A) to the keratin material,
(2) allowing the first composition (A) to act on the keratin material for a period of from about 1 to about 10 minutes,
(3) rinsing the first composition (A) out of the keratin material,
(4) applying the second composition (B) to the keratin material,
(5) allowing the second composition (B) to act on the keratin material for a period of from about 1 to about 10 minutes, and
(6) rinsing the second composition (B) out of the keratin material.

17. A multicomponent packaging unit (kit-of-parts) for treating keratinous material, comprising separately prepared
a first container containing a first composition (A) and
a second container containing a second composition (B), wherein the first and second compositions (A) and (B) are defined in claim 1.

18. The method according to claim 1, wherein the total content of all polymers presents in the first composition (A)—based on the total weight of the first composition (A)—is below about 0.01% by weight.

19. The method according to claim 1, wherein the total content of all polymers presents in the second composition (B)—based on the total weight of the second composition (B)—is below about 0.01% by weight.

20. The method according to claim 1, wherein the second composition (B) comprises—based on the total weight of the second composition (B)—the one or more amino acid and/or protein hydrolysate of compound (B1) in a total amount of about 0.1 to about 20.0% by weight.
